(19) United States
(12) Patent Application Publication
(10) Pub. No.: US 2023/0081374 A1
(43) Pub. Date: Mar. 16, 2023

(54) SEGMENTATION INSTRUMENT AND CONTROLLER

(71) Applicant: Eximis Surgical Inc., Louisville, CO (US)

(72) Inventors: Dirk Johnson, Louisville, CO (US); Kristin D. Johnson, Louisville, CO (US); William N. Gregg, Superior, CO (US); Steven C. Rupp, Arvada, CO (US); Steve Choi, Lafayette, CO (US); Armando Garcia, Longmont, CO (US); Hana Creasy, Westminster, CO (US); Chris Underwood, Broomfield, CO (US)

(21) Appl. No.: 17/895,801

(22) Filed: Aug. 25, 2022

Related U.S. Application Data
(60) Provisional application No. 63/237,025, filed on Aug. 25, 2021.

(51) Int. Cl. A61B 18/14

(52) U.S. Cl. CPC A61B 18/1482; A61B 2018/00601

(57) ABSTRACT

A tissue segmentation device comprises segmenting wires, a grasper, an introducer tube that is shaped and sized to allow introduction of the segmenting wires and the grasper into a patient incision, a specimen bag configured to be deployed through the introducer tube and into the patient incision, at least one actuator positioned adjacent a proximal end of the introducer tube and coupled to proximal portions of the segmenting wires and the grasper, and wherein the at least one actuator is configured for manipulating the grasper to grasp a tissue specimen prior to or during tissue segmentation, wherein manipulation of the grasper further enables pulling the tissue specimen into the segmenting wires for segmentation, positioning the tissue specimen such that it contacts the segmenting wires, and/or enabling placement of the tissue specimen in the bag.

### PRIORITY AND CROSS REFERENCE TO RELATED APPLICATIONS

The present application for patent claims priority to Provisional Application No. 63/237,025, entitled “Segmentation Instrument and Controller,” filed Aug. 25, 2021 and assigned to the assignee hereof and hereby expressly incorporated by reference herein.

This application is related to U.S. application Ser. No. 16/381,661, entitled “Tissue Specimen Removal Device, System and Method,” filed Apr. 11, 2019, U.S. Pat. No. 9,649,147 issued May 16, 2017 and entitled “Electrosurgical Device and Methods,” and U.S. Pat. No. 9,522,034 issued Dec. 20, 2016 and entitled “Large Volume Tissue Reduction and Removal System and Method,” the entire disclosures of which are hereby incorporated by reference for all proper purposes, as if fully set forth herein. The present application for patent is also related to U.S. Pat. Nos. 10,925,665; 10,603,100; and U.S. Pat. No. 10,873,164 entitled “Large volume Tissue Reduction and Removal System and Method”, “Electrosurgical Device and Methods”, and “Connector”, respectively, assigned to the assignee hereof and hereby expressly incorporated by reference herein.

While various novel features are described herein, they can be used alongside or in conjunction with the inventions and disclosures set forth in the patents mentioned above. Therefore, the relevant text, figures and other disclosure from these prior patents are included in the present disclosure for context, background, and where necessary, incorporation into aspects of the disclosure described herein.

### FIELD OF THE DISCLOSURE

The present disclosure relates generally to devices, systems, and methods for removal of biological tissue during surgical procedures. In particular, but not by way of limitation, the present disclosure relates to an instrument for segmenting tissue specimen and a connector for coupling components of a tissue segmentation and removal device.

### BACKGROUND

Current methods for removing large tissue specimens with minimally invasive procedures such as, but not limited to, hysterectomy, nephrectomy, and splenectomy are to use morcellators or to manually reduce the tissue size with radio frequency (RF) energy, mechanical cutting or fracture methods. These methods require a considerable amount of time and many sequential steps to complete. An alternative to the morcellator technique is to create a larger incision for the access port so that the tissue specimen can be removed in whole. Unfortunately, this approach leads to more patient pain and longer recovery times.

The description provided in the background section should not be assumed to be prior art merely because it is mentioned in or associated with the background section. The background section may include information that describes one or more aspects of the subject technology.

### SUMMARY

The following presents a simplified summary relating to one or more aspects and/or embodiments disclosed herein. As such, the following summary should not be considered an extensive overview relating to all contemplated aspects and/or embodiments, nor should the following summary be regarded to identify key or critical elements relating to all contemplated aspects and/or embodiments or to delineate the scope associated with any particular aspect and/or embodiment. Accordingly, the following summary has the sole purpose to present certain concepts relating to one or more aspects and/or embodiments relating to the mechanisms disclosed herein in a simplified form to precede the detailed description presented below.

For the purposes of this disclosure, and when referencing a direction of intended surgery, the terms “front” and “distal” shall refer to a side or direction associated with a direction of intended surgery (i.e., towards the patient body, inside the patient body), while the terms “back”, “rear”, or “proximal” shall be associated with the intended bracing of the grasper (i.e., towards the surgeon or the surgical team). For instance, FIG. 1 shows a grasping tong 1016 at the distal end and handles 1018 at the proximal end.

An aspect of the present disclosure provides a tissue segmentation device, comprising one or more segmenting wires; a grasper; an introducer tube having a proximal end and a distal end, wherein the introducer tube is shaped and sized to allow introduction of the one or more segmenting wires and the grasper into an incision in a patient; a specimen bag, wherein the specimen bag is configured to be deployed through the introducer tube and into the incision in the patient; at least one actuator positioned at or near the proximal end of the introducer tube, wherein the at least actuator is coupled to a proximal portion of the one or more segmenting wires and a proximal portion of the grasper, and wherein the at least one actuator is configured for manipulating the grasper to grasp a tissue specimen prior to or during tissue segmentation. In some implementations, manipulation of the grasper further enables one or more of (1) pulling the tissue specimen into the one or more segmenting wires for segmenting said tissue specimen, (2) positioning the tissue specimen such that it contacts the one or more segmenting wires, and (3) enabling placement of the tissue specimen in the specimen bag.

Another aspects of the disclosure provides a tissue segmentation device, comprising one or more wire loop spools, one or more segmenting wires, wherein at least a portion of each of the one or more segmenting wires is wound on one of the one or more wire loop spools, and a tensioning mechanism comprising at least one motor, wherein the at least one motor of the tensioning mechanism is coupled to the one or more wire loop spools and configured to provide an adjustable force to advance or retract the one or more segmenting wires via a corresponding wire loop spool.

Another aspect of the disclosure provides a tissue segmentation device, comprising a disposable portion comprising one or more wire loop spools, wherein a segmenting wire is wound around each of the one or more wire loop spools, and a reusable portion, the reusable portion comprising at least a tensioning mechanism assembly, wherein the tensioning mechanism assembly is configured to couple to each of the one or more wire loop spools, and wherein the tensioning mechanism assembly is further configured for applying tension to the one or more segmenting wires via rotation of the one or more wire loop spools.

In some implementations, the one or more segmenting wires comprise a plurality of segmenting wires, and wherein at least one of the plurality of segmenting wires is an active electrode configured to carry radio frequency (RF) energy.

In some implementations, the active electrode is a stationary electrode, and the grasper comprises the return electrode, and wherein the manipulation of the grasper comprises pulling the tissue specimen into the active electrode for segmentation of said tissue specimen.

In some implementations, the actuator is configured to expand the active electrode into a bulbous loop shape adjacent to, but not in contact with, a return electrode, and wherein the grasper comprises the return electrode.

In some implementations, at least a portion of the grasper is conductive, the grasper comprises a return electrode, the at least one active electrode comprises a single active electrode, and a surface area of the return electrode is greater than a surface area of the single active electrode.

In some implementations, the one or more segmenting wires comprises a plurality of segmenting wires, the plurality of segmenting wires shaped and sized to fit within an inner diameter of the introducer tube.

In some implementations, the plurality of segmenting wires comprise an expanded position and a retracted position, and wherein, when in the expanded position, the plurality of segmenting wires are configured to extend at an angle from the distal end of the introducer tube, and when in the retracted position, the plurality of segmenting wires are parallel or substantially parallel to each other and configured to retract into the distal end of the introducer tube.

In some implementations, when in the expanded position, the plurality of segmenting wires are configured to segment the tissue specimen upon one of (1) pulling the tissue specimen into the plurality of segmenting wires using the grasper, wherein the grasper comprises a return electrode, and wherein one or more of the plurality of segmenting wires comprise an active electrode, or (2) pushing the plurality of segmenting wires into the tissue specimen, wherein one or more of the plurality of segmenting wires comprise an active electrode.

In some implementations, the one or more segmenting wires comprises a plurality of segmenting wire loops, and wherein positioning the tissue specimen further comprises encircling at least a portion of the tissue specimen using the plurality of segmenting wire loops.

In some implementations, the tissue segmentation device further comprises a plurality of retractable tines configured to expand from and retract into the distal end of the introducer tube, wherein at least one of the plurality of tines is a return electrode and at least two of the plurality of tines are active electrodes, and wherein the return electrode is arranged opposing the active electrodes such that the return electrode does not contact the active electrodes.

In some implementations, the one or more segmenting wires include non-uniform surface features for gripping the tissue specimen.

In some implementations, the one or more segmenting wires comprise a plurality of segmenting wire loops, the tissue segmentation device further comprising an introducer tube having a proximal end and a distal end, wherein the introducer tube is shaped and sized to allow introduction of the one or more segmenting wires into an incision in a patient.

In some implementations, the tissue segmentation device further comprises a multi-lumen tube comprising a plurality of lumens or channels, the multi-lumen tube shaped and sized to fit within an inner diameter of the introducer tube, and a plurality of connector pins coupled to ends of the plurality of segmenting wire loops, wherein each of the plurality of connector pins is received within one lumen or channel of the multi-lumen tube.

In some implementations, the tissue segmentation device further comprises a connector for reducing or minimizing friction between the plurality of segmenting wire loops and the multi-lumen tube, wherein the connector is positioned at or near a distal end of the multi-lumen tube, and wherein the plurality of connector pins are positioned on a proximal portion of the connector.

In some implementations, the multi-lumen tube further comprises a rod, the rod shaped and sized to be received within a lumen or channel of the multi-lumen tube, and wherein a central axis of the rod is positioned at or near a central axis of the multi-lumen tube.

In some implementations, the tensioning mechanism further comprises one or more of a constant force spring, a constant torque spring, a pulley system, a cable drive, a winch system, one or more non-linear springs, a linear drive with rotational coupling, a linear drive with magnetic coupling, and an electromechanical drive, the electromechanical drive selected from a group consisting of a servo motor, a stepper motor, a direct current (DC) motor, and a linear actuator.

In some implementations, the tensioning mechanism further comprises at least one DC motor, and wherein each of the at least one wire loop spools comprises a slot that is shaped and sized to receive a rotating paddle from one of the at least one DC motor, and wherein each of the at least one DC motor is configured to provide an adjustable force to one of the one or more segmenting wires via a corresponding wire loop spool.

In some implementations, one of the at least one wire loop spools comprises a conductive metal disk and a drag strip connection for electrically coupling a radio frequency (RF) generator to a corresponding one of the segmenting wires via the DC motor.

In some implementations, the tensioning mechanism is coupled to a pneumatic system, the pneumatic system configured to generate pressure that is above a threshold for driving a translation force for advancing or retracting the one or more segmenting wires.

In some implementations, the tensioning mechanism assembly comprises at least a motor and a spring. In some implementations, the motor is a direct current (DC) motor and the spring is a constant torque or constant force spring.

In some implementations, the tensioning mechanism assembly comprises one or more motors, each of the one or more motors having a paddle that is configured to rotate when a voltage is applied to the corresponding motor.

In some implementations, each of the one or more wire loop spools comprises a slot that is shaped and sized to receive a paddle from one of the one or more motors, and wherein the rotation of the one or more wire loop spools is based at least in part on the rotation of the one or more paddles.

In some implementations, the one or more segmenting wires are pre-tensioned prior to coupling the tensioning mechanism assembly to the one or more wire loop spools, wherein pre-tensioning the one or more segmenting wires comprises manually or mechanically winding the one or more one or more wire loop spools.

In some implementations, prior to or during pre-tensioning, the one or more wire loop spools are prevented from rotating backwards, thereby preventing the one or more segmenting wires from advancing, based at least in part on an interaction of the one or more wire loop spools with the tensioning mechanism assembly.

In some implementations, the one or more wire loop spools comprises a wire loop spool, and wherein, the tensioning mechanism assembly comprises a direct current (DC) motor having a rotating paddle, and the wire loop spool comprises a first slot that is shaped and sized to receive the rotating paddle, a conductive disk having an opening that is shaped and sized to receive the first slot, a connecting element coupled to a segmenting wire of the one or more segmenting wires, a second slot that is shaped and sized to receive the connecting element, and a drag strip connection.

In some implementations, the segmenting wire of the one or more segmenting wires is coupled to the connecting element via a conductive cable or strand, and wherein the drag strip connection is coupled to one or more of the connecting element and the conductive disk.

In some implementations, the conductive disk is configured to receive a radio frequency (RF) signal from the DC motor, and wherein the drag strap connection is further configured to supply the RF signal to the segmenting wire via the connecting element.

In some implementations, each of the one or more segmenting wires is an active electrode configured to receive a radio frequency (RF) signal from a RF generator.

In some implementations, applying tension to each of the one or more segmenting wires comprises retracting a corresponding one of the segmenting wires.

In some implementations, the tissue segmentation device further comprises at least one controller, the at least one controller configured to control one or more of (1) a power output of a radio frequency (RF) generator, the RF generator configured to supply RF energy or power to the one or more segmenting wires, and (2) a torque or force applied by a force application mechanism of the tensioning mechanism assembly to each of the one or more segmenting wires. In some implementations, the controller is configured to control the torque or force applied by the force application mechanism based at least in part on determining one or more of (1) a rate of travel of the force application mechanism, (2) a distance of travel of the force application mechanism, (3) a rate of travel of each of the one or more segmenting wires, and (4) a distance of travel of each of the one or more segmenting wires.

In some implementations, the force application mechanism comprises a constant force spring configured to cause the one or more segmenting wires to apply a constant force to a tissue specimen, wherein the tissue segmentation device is configured to apply the RF power to the one or more segmenting wires while applying the constant force, and wherein each of the one or more segmenting wires comprises an active electrode.

In some implementations, the tensioning mechanism assembly comprises a direct current (DC) motor, and wherein the at least one controller is further configured to control a velocity of the DC motor based at least in part on controlling a current or a voltage used to drive the DC motor.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

FIG. 1 illustrates an example of a grasper comprising an integrated return electrode, according to various aspects of the present disclosure;

FIG. 2 illustrates an example of an actuator system comprising an active electrode and a grasper with an integrated return electrode, according to various aspects of the present disclosure;

FIG. 3 illustrates an example of a collapsible wire screen electrode in a collapsed position, according to various aspects of the present disclosure;

FIG. 4 illustrates the collapsible wire screen electrode of FIG. 3 in an expanded position, according to various aspects of the present disclosure;

FIG. 5 illustrates an example of an actuator system comprising a grasper and the collapsible wire screen electrode of FIG. 4, according to various aspects of the present disclosure;

FIG. 6 illustrates another example of a grasper for use in a tissue segmentation system, according to various aspects of the present disclosure;

FIG. 7 illustrates a perspective view of components of an actuator, according to various aspects of the disclosure;

FIG. 8 illustrates an electrosurgical device and system for detecting a distance of electrode travel, according to various aspects of the present disclosure;

FIG. 9 illustrates a detailed view of a detachable and/or reusable motor configured for use in a tissue segmentation system, according to various aspects of the present disclosure;

FIG. 10A illustrates an electrical connection mechanism for coupling a motor to an electrode wire loop spool, according to various aspects of the present disclosure;

FIG. 10B illustrates an exploded view of the electrical connection mechanism in FIG. 10A, according to various aspects of the present disclosure;

FIG. 10C illustrates an example of a wire loop retraction mechanism, according to various aspects of the present disclosure;

FIG. 11A illustrates an embodiment of a specimen removal bag system with the specimen bag open in accordance with various aspects of the invention;

FIG. 11B illustrates a connector housing and connectors for use in a tissue segmentation device, according to various aspects of the present disclosure;

FIG. 12A illustrates an example of an insertion tube and a multi-lumen tube for use in a tissue segmentation system, according to various aspects of the present disclosure;

FIG. 12B illustrates another example of an insertion tube and a multi-lumen tube for use in a tissue segmentation system, according to various aspects of the present disclosure;

FIG. 12C illustrates an example of an insertion tube and a multi-lumen tube having a stiffener rod for use in a tissue segmentation system, according to various aspects of the present disclosure;

FIG. 13 illustrates an example of a tissue specimen bag deployed inside a cavity of a patient and a grasper, according to various aspects of the present disclosure;

FIG. 14 illustrates another example of a tissue specimen bag deployed inside a cavity of a patient and a grasper, according to various aspects of the present disclosure;

FIG. 15 illustrates an example of a tissue removal system coupled to a radio frequency (RF) generator, according to various aspects of the disclosure;

FIG. 16 illustrates a tissue segmentation device, including a controller, according to various aspects of the disclosure;

FIG. 17 illustrates an example of a sensing device configured for use in a tissue segmentation device, according to various aspects of the disclosure;

FIG. 18 is a perspective view of a disposable lumen assembly and a reusable tensioning mechanism assembly, according to various aspects of the disclosure;

FIG. 19 illustrates a tissue segmentation device having disposable and reusable portions, according to various aspects of the disclosure;

FIG. 20A is a perspective view of a tissue segmentation device having a tensioning mechanism, according to various aspects of the disclosure;

FIG. 20B is a perspective view of the device in FIG. 20A with some components removed, according to various aspects of the disclosure;

FIG. 20C is a top view of some components of the device in FIG. 20A, according to various aspects of the disclosure;

FIG. 21 illustrates an example of a spring for tensioning segmenting wires, according to various aspects of the disclosure;

FIG. 22 illustrates an example of a dual return electrode positioned at an end of an introducer tube, according to various aspects of the disclosure;

FIG. 23 illustrates another example of a dual return electrode positioned at or near an end of an introducer tube, according to various aspects of the disclosure;

FIG. 24 illustrates an example of a dual return electrode positioned at an end of an introducer tube, according to various aspects of the disclosure.

### DETAILED DESCRIPTION

The present disclosure relates to devices, systems, and methods for removal of biological tissue during surgical procedures. In particular, but not by way of limitation, the present disclosure relates to an instrument for segmenting tissue specimen and a connector for coupling components of a tissue segmentation and removal device.

The word “exemplary” is used herein to mean “serving as an example, instance, or illustration.” Any embodiment described herein as “exemplary” is not necessarily to be construed as preferred or advantageous over other embodiments.

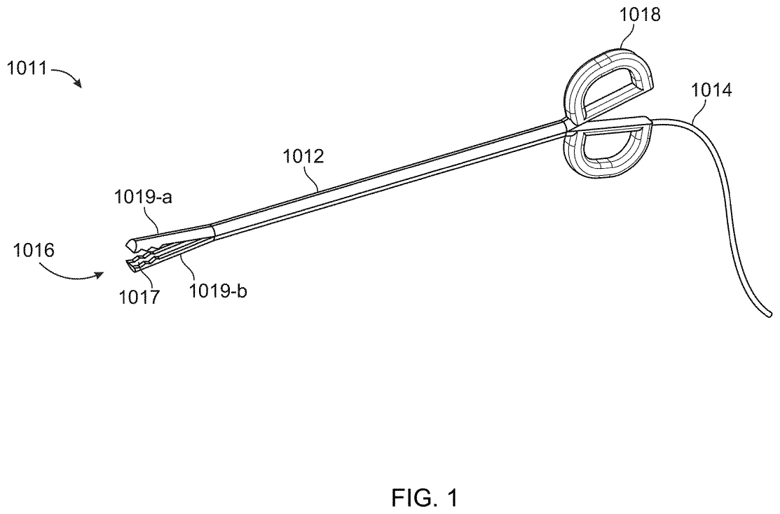

For the purposes of this disclosure, and when referencing a direction of intended surgery, the terms “front” and “distal” shall refer to a side or direction associated with a direction of intended surgery (i.e., towards the patient body, inside the patient body), while the terms “back”, “rear”, or “proximal” shall be associated with the intended bracing of the grasper (i.e., towards the surgeon or the surgical team). For instance, FIG. 1 shows a grasping tong 1016 at the distal end and handles 1018 at the proximal end. Furthermore, for the purposes of this disclosure, the terms “introducer tube”, “insertion tube”, and “distal tube” may be used interchangeably and may refer to a tube that is configured to enter the patient cavity through an incision and that is shaped and sized to permit one or more laparoscopic tools (e.g., grasping tong 1016) to be inserted into the patient incision during a surgical procedure. Furthermore, the term “outer tube” may refer to a tube that is shaped and sized to encase, for instance, a rolled-up containment bag assembly. In some cases, the outer tube is also shaped and sized to receive an inner tube, where the inner tube is used to push the rolled-up containment bag assembly out of the distal end of the outer tube, for instance, to unfurl the rolled-up bag. In some embodiments, the outer tube may be the same or different from the introducer tube. That is, in some cases, the outer tube comprising the rolled-up containment bag also serves as the introducer tube.

The present application for patent is related to U.S. Pat. No. 10,925,665 ('665 patent); U.S. Pat. No. 10,603,100 ('100 Patent); and U.S. Pat. No. 10,873,164 ('164 patent) entitled “Large volume Tissue Reduction and Removal System and Method”, “Electrosurgical Device and Methods”, and “Connector”, respectively, assigned to the assignee hereof and hereby expressly incorporated by reference herein. The present application for patent is also related to U.S. Publication No. 2019/0328377 ('377 Publication) entitled “Tissue Specimen Removal Device, System and Method,” assigned to the assignee hereof and hereby expressly incorporated by reference herein.

Increasingly, improvements in surgery techniques pertain to reducing the invasiveness of procedures. In particular, surgeons seek to perform “minimally invasive” procedures—meaning that incisions are limited to a particular size—whenever possible. However, many surgeries that can be performed almost entirely via very small incision sites end up requiring a last step that is very difficult to perform via a small incision site. That last step is the removal of excised tissue. Removing large portions of tissue, such as entire uteri, large portions of kidneys, or cancerous tumors, for example, creates a number of logistical challenges. The previous disclosures referenced throughout this present disclosure describe various devices, systems, and methods for segmenting these large pieces of tissue within a specimen bag while still inside the patient. Current approaches allow for the tissue to be segmented into small enough pieces that they can be pulled out one by one through the small incision site.

Several factors can make this process time consuming, difficult, messy, and/or lead to a patient risk. For example, if a portion of the tissue is calcified, currently available cutting devices may take a long time to cut through that portion. In such cases, bringing the tissue close to the top of the specimen bag and cutting it as the tissue is being extracted can take an hour or more, and may require many hands and tools in the area. If the tissue and specimen bag must be manipulated and handled excessively, the opening of the bag may slip back into the incision site. This can be particularly high risk to a patient if the tissue specimen is a cancerous tumor because such specimens often contain liquid that can spill and spread cancer cells within the patient's body. The present disclosure provides devices, systems, and methods that improve the ease, safety, and efficiency of segmenting a tissue specimen within a specimen bag.

One type of existing specimen bag or containment component system is a flexible material that is rolled or folded by a surgeon, attending surgeon and/or scrub nurse so that it can be inserted through the trocar or incision site and then opened once inside the patient's body. In this type of system, the surgeon first excises the tissue to be removed, and then manipulates the bag opening with laparoscopic tools in order to place the tissue specimen within the bag. After capture of the tissue, the bag opening is raised with laparoscopic graspers (e.g., grasper 1011 in FIG. 1) and led out of the incision site to be secured externally by the surgeon by hand or with the addition of Kelly clamps or snaps.

Some of these types of specimen bags incorporate a polymer ring that is formed or attached to the top of the bag to keep the bag opening biased to a fully open position. This polymer ring can help hold the exteriorized bag open and in an appropriate place so that it does not fall back into the peritoneum or other surgical site of a patient.

Another common type of specimen bag or containment component system uses a bag that is typically placed within a cannula or lumen for insertion into the peritoneum through a trocar or incision site and the specimen bag advances beyond the cannula to access the opening.

Many specimen bag systems use a mechanical means to bias the bag opening to an extended position to assist the surgeon in placing the tissue specimen within the bag. Such systems may comprise a formed metal ring with a spring bias attached to the top of the specimen bag so that the spring bias opens the top of the specimen bag when it is outside of the cannula. Most of the systems that use a metal ring of this type also incorporate a string or suture material as a drawstring to close the bag opening for exteriorization. In these devices, the string may remain outside of the patient's body and be pulled to seal the bag. This string closes the opening while the metal ring is retracted back into the cannula leaving the bag free from the cannula and metal rings and also leaving the bag within the incision site after the cannula and metal ring are withdrawn. Then, the surgeon can use the string to pull the bag opening through the incision site. Other systems use a string or suture material as a drawstring that closes the bag opening and while doing so, tears the bag away from the metal ring, leaving the bag free from the metal rings and cannula. The string is then used to retrieve the bag opening through the incision site.

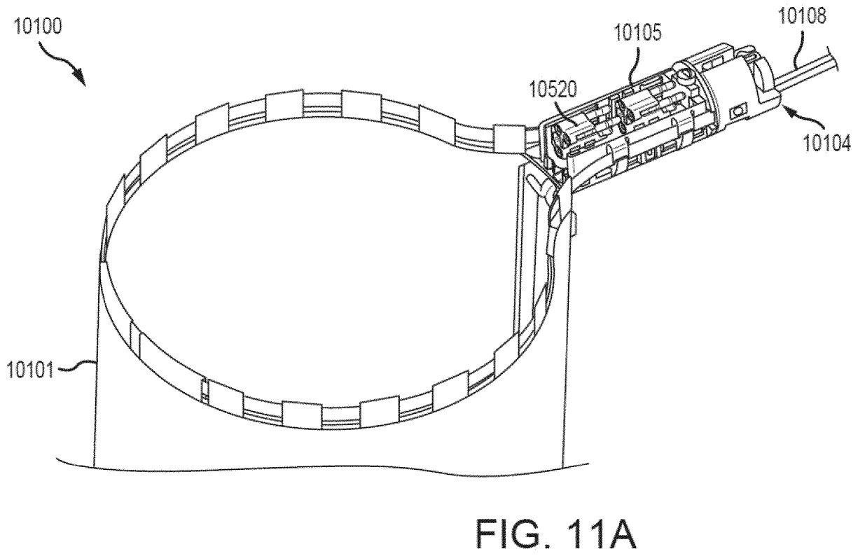

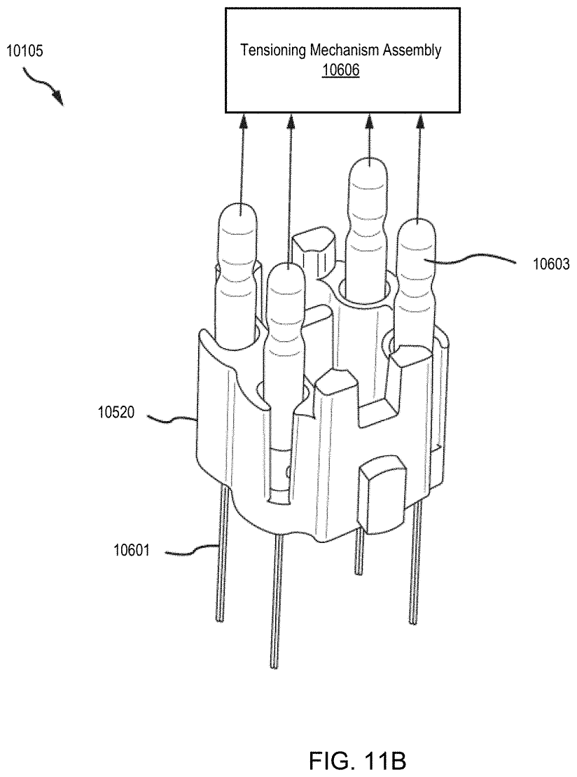

In one non-limiting example, a metal ring subassembly comprised of two halves of a metal ring may be utilized to aid in the closing of the formed metal ring attached to the top of the specimen bag, as shown in FIG. 11A. In some embodiments, the distal interface point between the two metal ring halves may be connected by a flexible member. In some circumstances, this flexible member may allow the spring biased metal ring to be compressed into a nearly flat configuration such that the two halves of the spring biased metal ring are parallel or substantially parallel to each other. According to aspects of this disclosure, this flexible member may be created in a variety of methods that may facilitate in retention of the distal ends of the two metal ring halves. Some non-limiting examples of manufacturing techniques utilized for the flexible member may include manufacturing the flexible member from a flexible film (i.e., heat shrink), using a machined subassembly with an incorporated flexible hinge at the distal ring tip, or using an injection molded living hinge feature (i.e., an integral hinge made from the same material as the two pieces it connects) which allows the metal ring assembly to hinge freely at this distal tip over the life of the product. In some cases, for instance, with the metal ring subassembly in the compressed state, the containment bag subassembly may be configured to be rolled into a smaller diameter configuration which may facilitate placement through a patient incision. In some embodiments, this compressed and/or rolled containment bag assembly may be loaded into an outer tube (e.g., a thin-walled outer tube) which may aid in one or more of: product shipment, bag management during loading and/or deployment through a patient incision.

As previously described, the currently available specimen retrieval pouches are designed to contain tissue while a surgeon loads and subsequently exteriorizes the specimen bag. The Tissue Specimen Removal system described in the patents mentioned and incorporated above utilize tissue segmentation devices comprising wires, a return electrode, and other components. The Tissue Specimen Removal system of the present disclosure may integrate various tissue segmentation device components—for example, segmenting wires and a return electrode—and further include one or more “connectors.” The term “tissue segmentation device components,” or simply “segmenting components,” may refer to any type of cutting device that is configured to physically cut tissue. Often, these segmenting components comprise individual wires or wire loops, which cut tissue by being drawn through it by mechanical force, or with the assistance of RF energy, or with a combination of the two. However, any segmenting components described herein may include those referenced in each of the patents incorporated above, any referenced throughout this disclosure, or any other types of tissue cutting device known or yet to be created. In many embodiments, these segmenting components may be integrated into the specimen bag of the present disclosure prior to being deployed inside a patient. Examples of such specimen bags having integrated segmenting components (e.g., segmenting wire loops) are described later in this disclosure.

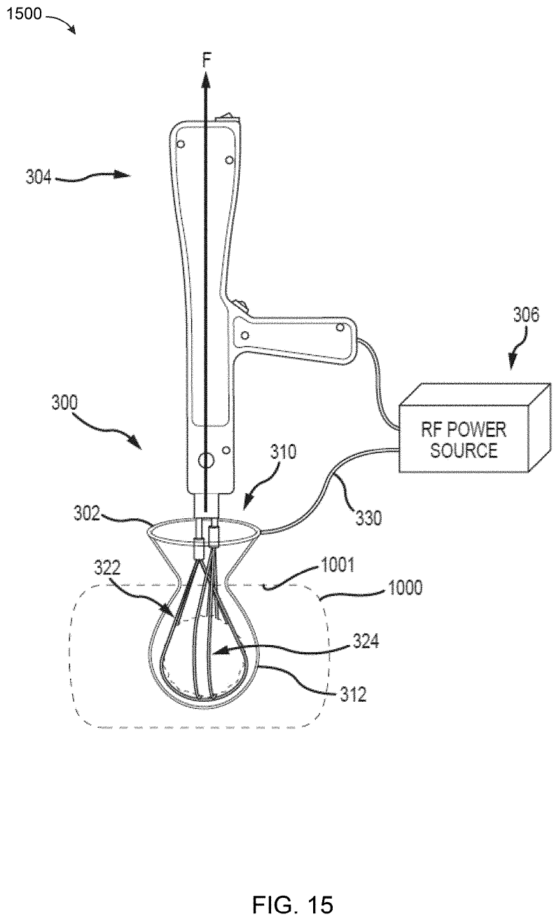

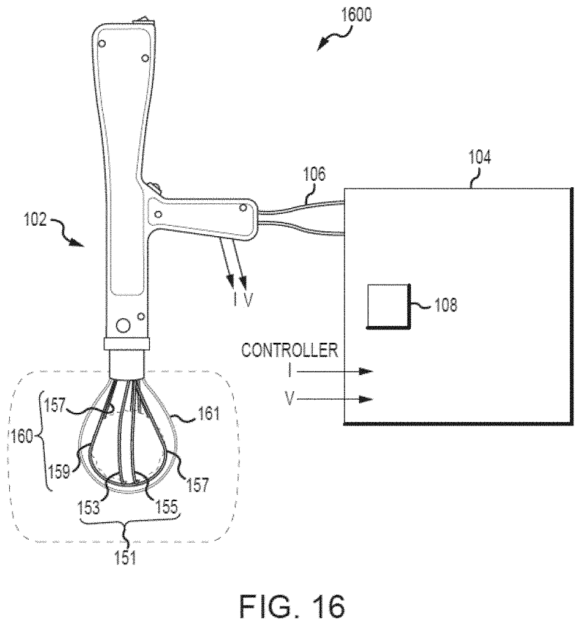

In one exemplary application, an advanced electrosurgical system may be provided. The system may be configured to perform some or all of the functions, such as tissue segmentation and/or removal, described in Applicant's International Application PCT/US 15/41407, entitled Large Volume Tissue Reduction and Removal System and Method, filed on Jul. 21, 2015, and having a priority date of Jul. 22, 2014, the entire contents of which are incorporated herein by reference for all purposes, as if fully set forth herein. The system may include an electrosurgical device and a generator (e.g., RF generator or power source 306 in FIG. 15) coupled together by a number of leads. In some cases, the generator (e.g., RF power source 306) may include a controller (e.g., shown as controller 108 in FIG. 16). Except as where otherwise stated herein, the term “segmentation device” shall be understood to include a device for dividing tissue, and may include a mechanical segmentation action, and/or an electrosurgical dissection action, for example a bipolar segmentation action, or a monopolar action.

In some cases, tissue specimen removal systems may be configured to reduce a large volume tissue specimen in size so that smaller pieces can be removed through an access port in the patient during minimally invasive surgery. In some cases, tissue specimen removal systems may employ a device, for instance, for introducing and deploying a specimen bag to capture and contain the tissue specimen during the procedure. Tissue specimen removal systems may also employ one or more RF electrosurgical generators. In some examples, the device may be adapted to segment the tissue specimen through RF energy-charged wires (e.g., wires 322 in FIG. 15), where the RF energy is received from the one or more RF electrosurgical generators (or simply, generators, such as RF power source 306 in FIG. 15). In some cases, the generator(s) may be set at the nominal power setting needed for tissue segmentation. The range of power settings may be determined based on the exposure size, or surface area between the tissue specimen and RF cutting wire. In one non-limiting example, the RF power used for tissue segmentation may be in the range of 60 to 400 Watts. In some instances, the RF energy or power is applied in a bipolar fashion, which helps prevent the current from being delivered to adjacent tissue structures. Containment of the tissue specimen in an insulative specimen/containment bag (e.g., container 312 in FIG. 15) may serve to add additional electrical isolation of the tissue specimen from the rest of the patient. In some embodiments, the RF generator may provide adjustments of amplitude or duty cycle of the output current, based at least in part on the current delivery and impedance observed during initiation and sustainment of the cut. In some cases, tissue specimen removal systems may utilize one or more connectors for connecting the RF electrosurgical generators, the tensioning mechanism, and other applicable components of the segmentation instrument to the segmenting electrodes/wires.

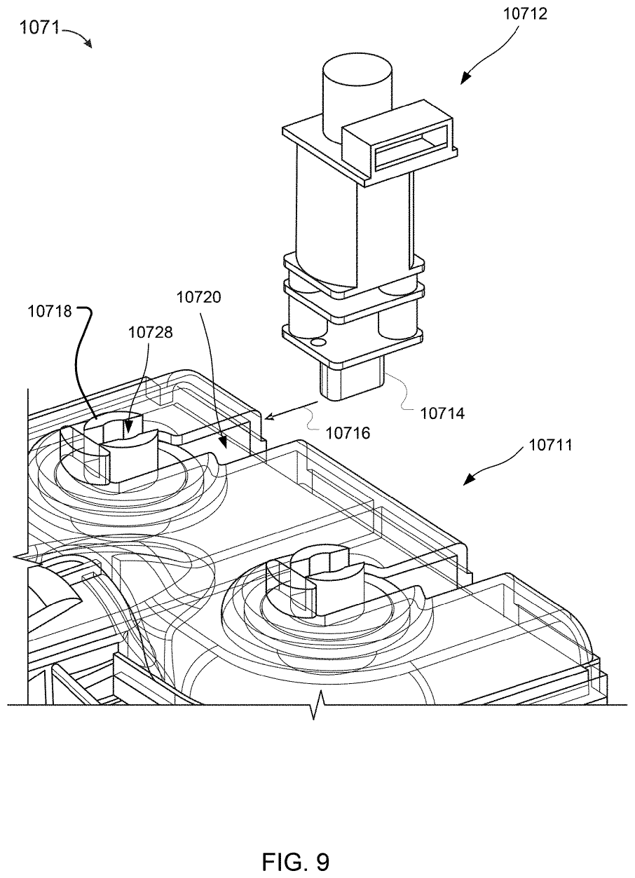

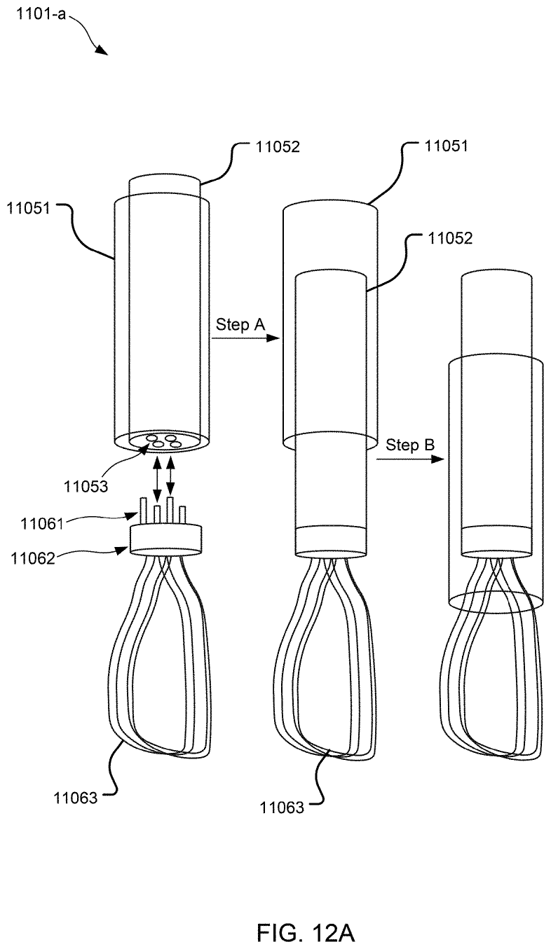

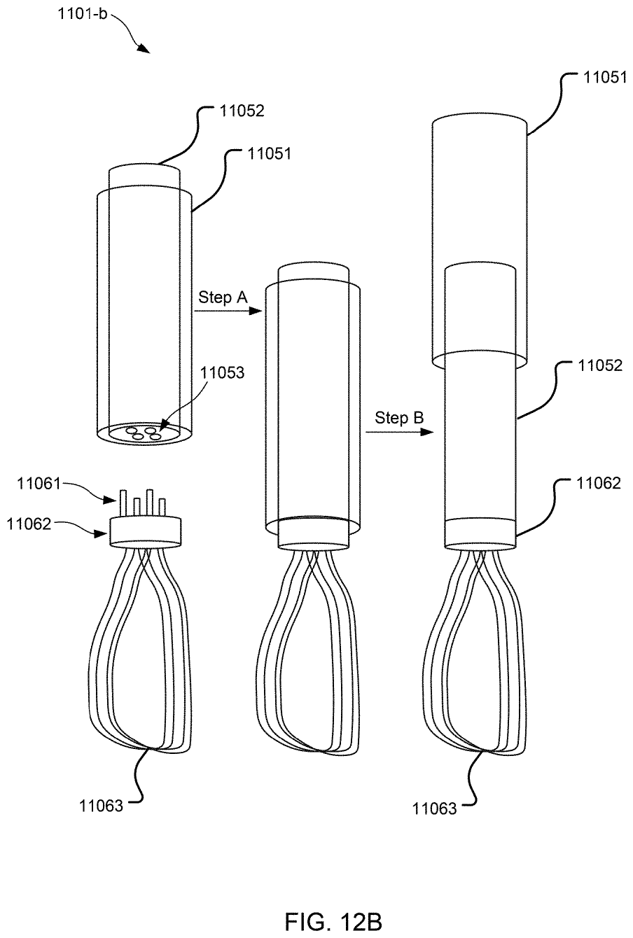

The term “connectors” may refer either to a connector housing (e.g., connector housing 10520 in FIG. 11A, connector 11062 in FIG. 12A) comprising one or more connector pins (e.g., connector pins 10603 in FIG. 11B, pins 11061 in FIG. 12A), or to individual connector pins themselves. The “connector pins” may also be referred to as “connector portions.” These connectors, such as connectors 11062 in FIGS. 12A-12C, attach to, at one end, segmenting components, such as wires 11063, within the specimen bag. The connectors are configured to allow later connection of a separate portion of a segmentation device. For ease of reference and differentiation between tissue segmentation device components, and this separate, connectable portion, the latter may be referred to herein as “connectable (tissue segmentation) equipment” or “a piece of connectable equipment.” For example, the connectable equipment may be a tensioning mechanism assembly, such as tensioning mechanism assembly or reusable portion 1071 in FIG. 9 or tensioning mechanism assembly 10606 in FIG. 11B. In some cases, the tensioning mechanism assembly may be configured to tension the segmenting components (cutting devices or segmenting wires) against the tissue specimen in preparation for drawing them through the tissue. The connectable equipment, in embodiments, may apply the required force and RF energy to the segmentation components and carry the return current back to the RF generator (e.g., RF power source 306). As such, a specimen bag of the present disclosure, which integrates connectors, segmentation wires, and a return electrode may have additional components not required for passive specimen retrieval pouch applications, as the dividing of tissue in those instances is done by the surgeon using separate tools not integrated with or connected to the bag.

In devices of the present disclosure, which comprise specimen removal bags that may be connected with connectable tissue segmentation equipment (e.g., a tensioning mechanism assembly), the components associated with the connectors are not required for the loading of the tissue, nor are they required during exteriorization. The devices and systems of the present disclosure includes these connectors because it is highly advantageous to integrate the one or more mechanisms for connection of tissue segmentation equipment (i.e., the connectors) into a tissue specimen collection bag itself. In particular, when collected tissue specimens need to be segmented while retained inside a specimen bag, it can be advantageous for a surgeon to be able to connect the segmenting components (e.g., segmentation wires or other cutting devices) quickly and easily to connectable tissue segmenting equipment (e.g., an RF powered tensioning device). Being able to activate and use the segmenting components quickly can save valuable time in critical moments after tissue mobilization. In some embodiments, the segmenting components comprise a plurality of wire loops integrated with the bag. Having the ends of these segmenting wires managed and out of the way, but then readily accessible once needed, is highly desirable. This can reduce the time spent retrieving additional instruments and reduce risks associated with setting equipment down and picking it up multiple times. Therefore, the integrated connector system of the present disclosure provides several conveniences and advantages.

### Introducer Tube

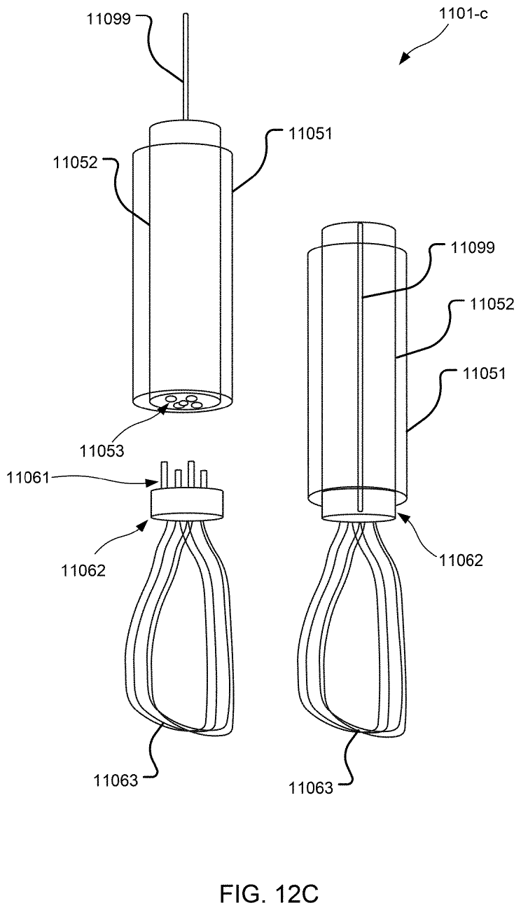

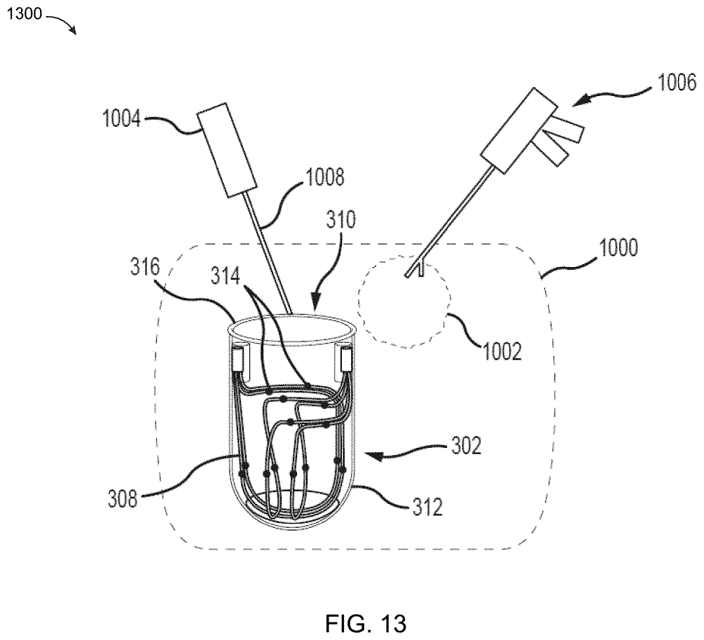

Illustrated in FIG. 13 is a tissue specimen retrieval device 1300 having a retrieval bag 302 deployed inside a cavity 1000 of a patient, according to various aspects of the disclosure. The retrieval bag 302 is shaped and sized so to receive a tissue specimen 1002 that is being surgically removed from the cavity 1000. Those of skill in the art will understand how to select the appropriate sizing of the retrieval bag 302 in relation to the particular tissue specimen 1002 being removed.

In the embodiment shown, the retrieval bag 302 has a container 312 with an entry 310, and a plurality of electrodes 308 disposed in the container 312 in a manner that will be described in further detail in subsequent portions of this disclosure. The container 312 may be flexible and deployable through a standard surgical tube, such as a cannula or lumen, as is known in the art. In some embodiments, a fastener 314 or a plurality of fasteners 314 may be provided to fasten the electrodes 308 (e.g., temporarily or permanently) to the container 312 in a desired configuration.

A spring-biased ring 316 may be provided at the entry 310 of the retrieval bag 302 to ease the opening of the retrieval bag 302; however, those of skill in the art will understand that this is not necessary to practice the invention. In some embodiments, the container 312 and the fasteners 314 are configured to be deployed through a tube, such as through a deployment instrument 1004, into the cavity 1000 and allowed to spring into place.

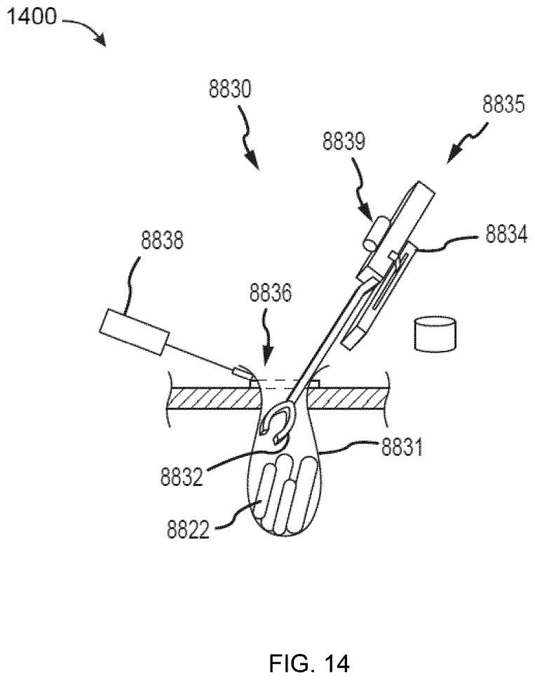

After the retrieval bag 302 is in place, a grasper 1006 (also shown as grasper 1011 in FIG. 1, grasper 8832 in FIG. 14), or another applicable means known in the art, may be provided to manipulate the specimen 1002 into the retrieval bag 302 prior to removal from the patient. Those of skill in the art will understand how the surgical team might loosen the specimen 1002 and move it into the retrieval bag 302.

FIG. 15 illustrates an example of a tissue removal system 1500 coupled to a radio frequency (RF) generator, according to various aspects of the disclosure. In some embodiments, and as illustrated in FIG. 15, a proximal force F may be applied to the electrodes 322 to initiate and/or maintain a tissue segmentation operation. In some cases, the electrode 322 may be active electrode wires and may be similar or substantially similar to the electrodes 308 described in relation to FIG. 13. Those of skill in the art will understand that an opposing force is necessary to maintain the actuator 304 and retrieval bag 302 in a stable position.

In some embodiments, portions of the retrieval bag 302 containing the specimen 1002 and electrodes 308 are configured to not contact the interior wall 1001 of the cavity 1000. In some embodiments, a distal insertion tube (e.g., insertion tube 11051 in FIG. 12A) is provided, against which the specimen 1002 may abut while the electrodes 322 are being pulled through the specimen 1002. In some embodiments, an additional thermal barrier (not shown) is provided in a wall of the retrieval bag 302 or on an exterior surface of the retrieval bag 302 so that any contact with the cavity 1000 will be protected from thermal damage. The thermal barrier may include a thermally insulative layer or a feature that can be inflated with air or a fluid. In some embodiments, the surgeon may use a laparoscopic camera to visually ensure that no contact is being made with the interior body cavity 1000.

In some embodiments, after the exteriorizing of the retrieval bag 302, an actuator 304 may be coupled to the proximal portions 320 of the electrodes 322. As will be understood by those skilled in the art, a generator 306, such as a radio frequency (RF) power source, may be coupled to the actuator 304, and a return electrode 330 may be coupled to the retrieval bag 302 if one was not previously provided. The tissue removal device 300 illustrated in FIG. 15 is in a ready-state for tissue segmentation, in accordance with one or more implementations.

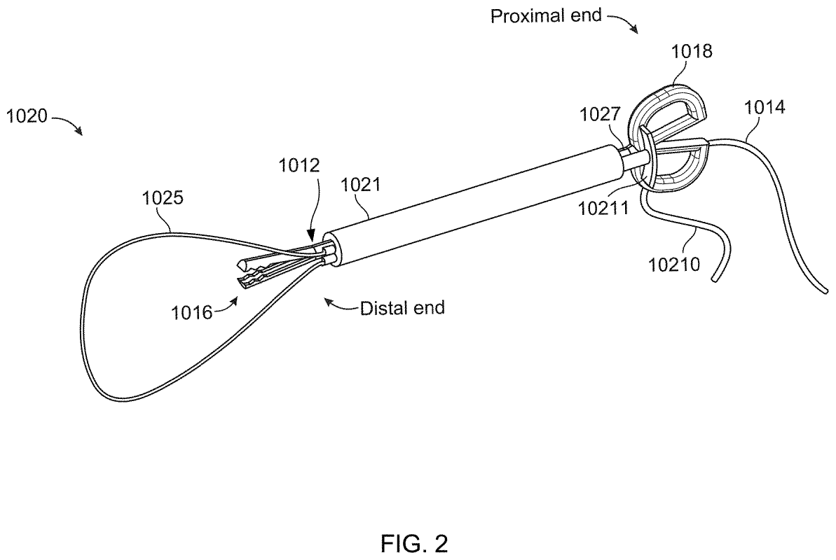

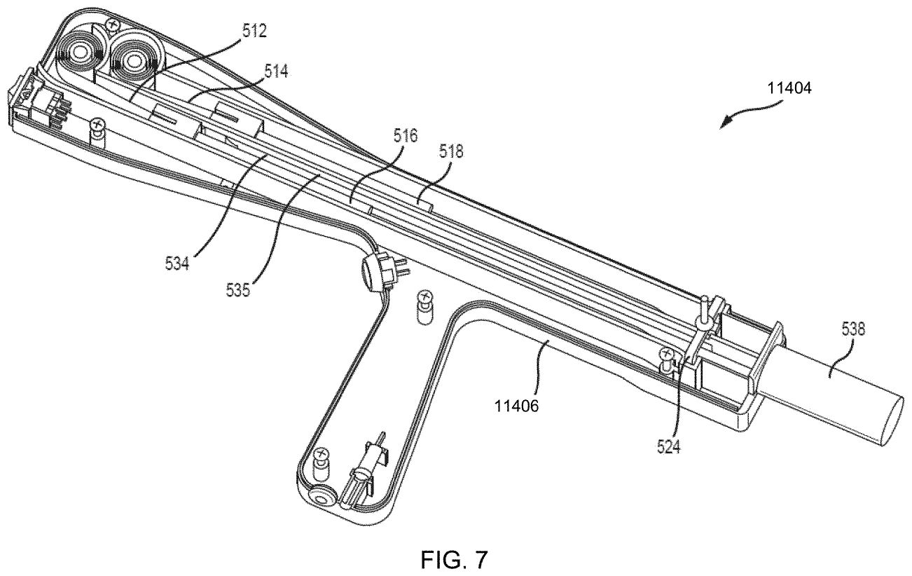

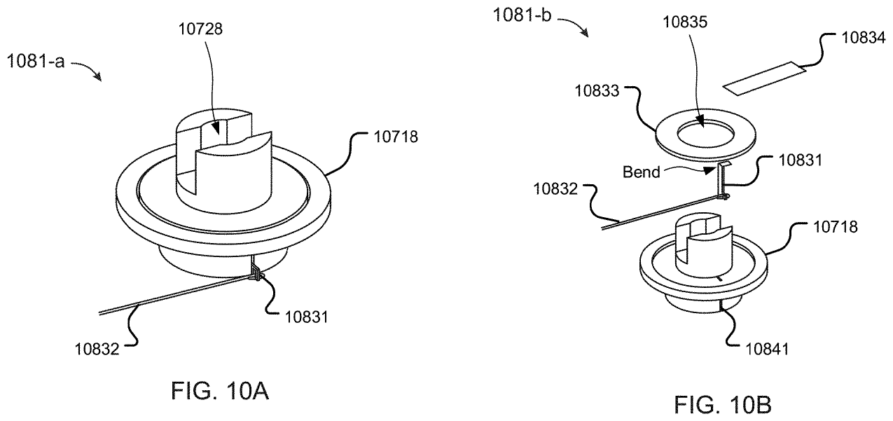

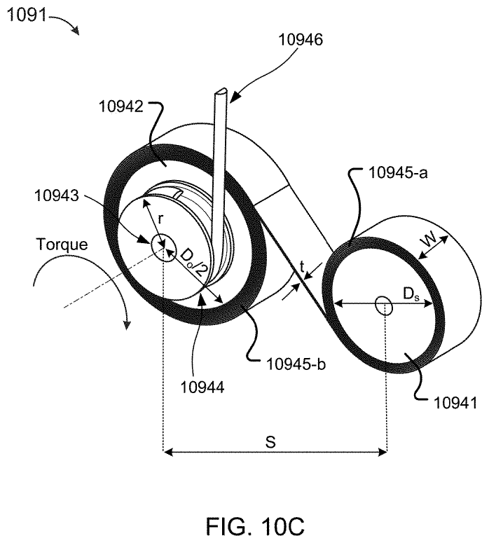

FIG. 7 illustrates a perspective view of components of an actuator 11404, according to various aspects of the disclosure. The actuator 11404 may be similar or substantially similar to the actuators described herein, including at least actuator 304 in FIG. 15. As seen, the actuator 11404 comprises an actuator housing 11406, a distal insertion tube 538, a first spring 512 of a first pull assembly (e.g., shown as pulley 10944 in FIG. 10C), a second spring 514, a first connector rod 516, a second connector rod 518, a power strip 534, a spring separation wall 535, and a spring pretension latch 524. In some embodiments, the actuator 11404 comprises a housing 11406 supporting one or more pull assemblies and a handle (e.g., handles 10211 and/or 1018 in FIG. 2) for assisting the user in controlling a position of the actuator 11404. In some cases, the first pull assembly is configured to apply a first force F1 on a specimen prior to and/or during a segmentation procedure, such as by way of a first electrode or first crimped set of electrodes. A second pull assembly may be configured to apply a second force F2 on the specimen prior to and/or during the segmentation procedure, such as by way of a second electrode or second crimped set of electrodes. The first force F1 may be applied or commenced prior to commencing application of the second force F2. The first force F1 may be completed prior to commencing application of the second force F2. The first force F1 may continue through at least a portion of an application of the second force F2. The magnitude of the first force F1 and the second force F2 may be controlled and varied in a manner discussed in other sections of this disclosure. That is, the forces F1, F2 may effect proximal forces on the specimen that vary during a segmentation procedure. While not necessary, the first force F1 and the second force F2 may have the same or similar magnitude, in some embodiments.

The first pull assembly may include the first spring 512, the first spring 512 coupled to a first connector rod 516 by way of a first spring-connector rod block (not shown). In some embodiments, the first spring 512 (and/or a second spring 514) may be a linear spring. Alternatively, the first and second springs may be constant torque springs, further described below in relation to FIG. 10C. The first connector rod 516 may be coupled or configured to couple to the first set of electrodes. Further, the second pull assembly may include the second spring 514 coupled to the second connector rod 518 by way of a second spring-connector rod block (not shown). The second connector rod 518 may be coupled or configured to couple to the second set of electrodes. In some examples, the spring holder or spring pretension latch 524 of the actuator 11404 may help maintain the springs 512, 514 in a tensioned state prior to a segmentation procedure. In some embodiments, the power strip 534 may be used to apply power to the electrodes (e.g., first, second set of electrodes). In some cases, the power strip 534 may be fixed and insulated within the housing 11406 so as to provide a separating wall between components of the first, second pull assemblies. In this example, the power strip 534 is aligned or attached to the spring separation wall 535.

In some examples, a distal insertion tube 538 may be provided to allow the actuator 11404 to be inserted into a laparoscopic opening, and the length of the tube 538 is such that with the tube 538 fully inserted into the patient, the specimen 1002 and electrodes 308 will remain out of contact with the interior of the cavity 1000, which may be the abdominal or thoracic wall. The distal end of the insertion tube 538 may be rounded, and/or include a lubricious material (e.g., shown as lubricious connector 11062 in FIG. 12A) to facilitate passage of the electrodes 308 between the insertion tube 538 and the specimen 1002. In some embodiments, the distal end of the insertion tube 538 may have openings or may be composed of a compliant material to facilitate wire movement. The proper instrument insertion length may be dictated by the instrument size proximal to the distal insertion tube. The distal insertion tube 538 may also have an inflatable feature, for example, on the proximal end near the actuator 11404, that sits between the specimen 1002 and the inside cavity wall 1001 to further prevent the wires or electrodes from contacting the patient's body wall during retraction of the electrodes 308. In some embodiments, an additional thermal barrier (not shown) is provided in a wall of the retrieval bag 302 or on an exterior surface of the retrieval bag 302 so that any contact with the cavity 1000 will be protected from thermal damage. The thermal barrier may include a thermally insulative layer or a feature that can be inflated with air or a fluid. In some embodiments, the surgeon may use a laparoscopic camera to visually ensure that no contact is being made with the interior body cavity 1000.

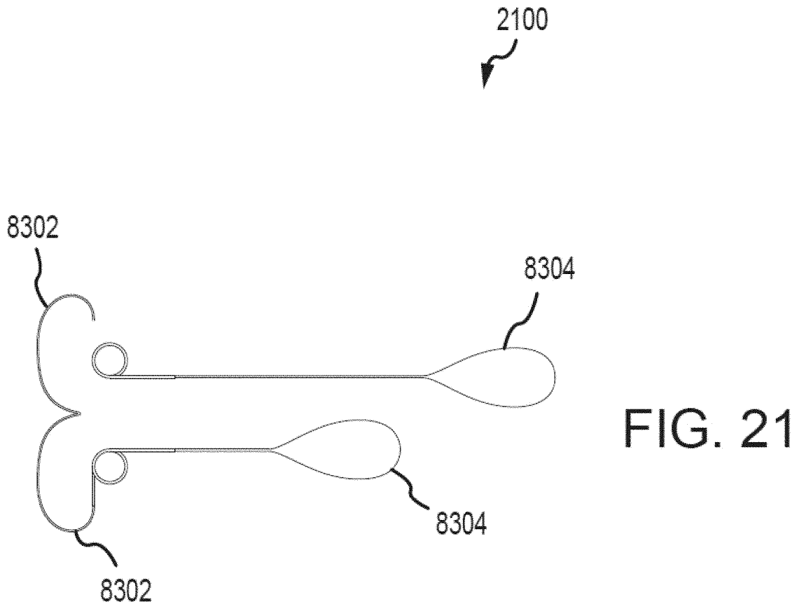

In some cases, the segmenting procedure incorporates an introducer tube (also referred to as intro tube), such as, introducer tube 1021 in FIG. 2. This tube, either independent or as a part of the segmenting instrument, may be placed through the opening of a loaded and exteriorized bag. In some cases, the segmenting wire loop(s), such as, segmenting wire loop 1025 in FIG. 2 or segmenting wire loop 8304 in FIG. 21, may be configured to pass through an internal channel or lumen of the tube, further described in relation to FIGS. 2 and 12A-13. In some cases, the distal end of the introducer tube may be placed proximal to the tissue to be segmented, for instance, inside the patient peritoneal cavity, but distal of the patient peritoneum. In some circumstances, this distal end of the intro tube may serve to provide a counterforce against the tissue and/or the segmenting wire loops while they are being retracting through the introducer tube. Additionally, or alternatively, this intro tube may facilitate in protecting one or more of the patient incision, the surrounding tissue, and the specimen bag being passed through the patient incision for the duration of the segmentation procedure. In some examples, the intro tube may be shaped and sized so that it may be placed within the lumen of a trocar. In some other cases, the bag may be designed to be integrated or coupled to a trocar.

In some embodiments, a single active electrode may be utilized to divide the tissue specimen completely independent of a specimen bag. Further, in some examples, the return electrode may be included as a feature on the distal end of the actuator introducer tube. In some embodiments, the return electrode may be a portion of a grasper used to hold the tissue specimen during segmentation, further described below in relation to FIGS. 1 and 2. Alternatively, the return electrode may include the entire conductive grasper. In yet other cases, the return electrode may be a conductive feature in the interior surface of the grasper, which serves to prevent accidental contact of the return electrode with the surrounding active electrode(s).

In some cases, the active wire loops may be configured to extend down a portion of the actuator shaft or introducer tube to deploy an active electrode loop (e.g., active electrode loop 1025 in FIG. 2) distal to the actuator. In some examples, one or more features or mechanisms on the actuator may be used to encourage the wire to expand distally into a bulbous loop shape, also described below in relation to FIG. 2. In some embodiments, the grasper or the wire loop may comprise an articulating feature/mechanism for repositioning the tissue specimen, which enables the wire loop to segment the tissue specimen at the intended location. In such cases, the wire loop (e.g., now encircling a portion of the tissue specimen) may be retracted by manual, mechanical, or electromechanical means to encase a portion of the tissue specimen. Additionally, or alternatively, RF energy may be used in conjunction with the mechanical force to pull the wire loop through the tissue specimen and divide the tissue. It should be noted that, this process may be repeated as needed to divide the tissue specimen into smaller chunks or segments. In some circumstances, RF energy may not be needed for smaller, or less dense tissue specimens that are to be divided. In other words, mechanical means may suffice for dividing smaller or less dense tissue specimens. It should be noted that, the approaches discussed above may also be used for multiple wire loops encircling the tissue specimen.

In some embodiments, the single wire loop cutter (e.g., active electrode loop) described above may be used as a disposable product for one division or for multiple divisions within one surgical procedure. In some other cases, the single wire loop cutter may be recleaned and sterilized between procedures and may be configured to be used for multiple procedures. In the latter cases, the functional limitation may depend on the integrity of the wire loop for multiple RF activations. In some embodiments, the wire loop may comprise one or more spools on each end of the wire loop for storing extra lengths of wire, further described below in relation to FIGS. 9-10C. In such cases, after RF activations, the extra wire on the spools may be advanced, for instance, to spool up the used wire portion and leave an unused portion of the spooled wire for subsequent segmentations. While not necessary, in some examples, the wire spools could be replaced as refillable cartridges for subsequent uses.

FIG. 1 illustrates an example of a grasper 1011 incorporating a return electrode, according to an embodiment of the disclosure. As seen, the grasper 1011 has a proximal end and a distal end, where the grasper 1011 comprises one or more handles 1018 and a return electrode cable 1014 at the proximal end and grasping tongs 1016 at the distal end. In this example, the grasper 1011 also comprise a tube 1012 positioned between the grasping tongs 1016 and the handle 1018. The tube 1012 may comprise a non-conductive outer surface and a hollow interior. In some embodiments, at least a portion of the return electrode cable 1014 may be received within the tube 1012. Further, the tube 1012 having the non-conductive outer surface may help protect the return electrode cable 1014, for instance, by minimizing or reducing the likelihood of an electrical short between the return electrode cable 1014 and the active electrodes (not shown in FIG. 1 but shown as active electrode wire loop 1025 in FIG. 2). In some embodiments, at least a portion of the distal end of the grasper 1011, for instance, the grasping tongs 1016, may be conductive. Further, the proximal end of the grasper, for instance, the handles 1018, may be non-conductive or composed of an electrically insulative material (e.g., plastic, a polymer, stiff rubber, to name a few non-limiting examples). In this way, the user (e.g., surgeon) and/or patient may be isolated from RF energy conduction.

In some cases, only a portion of the grasping tongs 1016 may be electrically coupled to the return electrode cable 1014. As seen, the grasping tongs 1016 comprise a first jaw 1019-a and a second jaw 1019-b opposing the first jaw. In one non-limiting example, the grasping tongs 1016 may include a conductive feature on an interior surface 1017 of the first and/or second jaws 1019. As noted above, this may serve to prevent accidental contact with the surrounding active electrodes (e.g., active electrode 1025 in FIG. 2). In another example, the first and/or the second jaws 1019 of the grasping tongs 1016 may be electrically coupled to the return electrode cable 1014 and may serve as the return electrode. In some other cases, only a portion of the jaws 1019, or only one of the two jaws 1019, may serve as the return electrode. It should be noted that, the examples listed above are not intended to be limiting and different return electrode configurations are contemplated in different embodiments.

FIG. 2 illustrates an example of a tissue segmentation device 1020, according to various aspects of the disclosure. The tissue segmentation device 1020 implements one or more aspects of the return electrode grasper 1011 previously described in relation to FIG. 1. As seen, the tissue segmentation device 1020 comprises a proximal end and a distal end, the proximal end having one or more handles 1018, a push/pull handle 10211, and a plurality of cables 1014, 10210. In some cases, one of the cables (e.g., cable 1014) is a return electrode cable and the other of the cables (e.g., cable 10210) is an active electrode cable. The tissue segmentation device 1020 further comprises an introducer tube 1021 having a first internal diameter, where the first internal diameter is of sufficient size to receive the grasper (i.e., the grasper 1011 including its tube 1012) and an inner tube or lumen for passing the wire loop 1025 through the patient incision. In some cases, the wire loop 1025 comprises an expanded position and a retracted position. In the expanded position, the wire loop 1025 expands or extends from the distal end of the introducer tube and comprises a bulbous loop shape, as depicted in FIG. 2. Furthermore, in the retracted position (not shown), the wire loop 1025 is configured to collapse and retract back into the distal end of the introducer tube 1021. In one non-limiting example, a user (e.g., surgeon) may manipulate the push/pull handle 10211 to expand or retract the wire loop 1025. The tissue segmentation device 1020 further comprises a tube 1012 positioned between the grasping tongs 1016 and the handles 1018. The tube 1012 and the grasping tongs 1016 may be similar or substantially similar to the ones described above in relation to FIG. 1. In some cases, the tissue segmentation device 1020 or actuator may include one or more features (e.g., guide features 1027 at the proximal end) to guide the integrated return grasper comprising the tube 1012 and grasping tongs 1016 such that its travel path is generally or always separated from the active electrode (e.g., wire loop 1025 in FIG. 2). In some cases, the active electrode wire(s), such as wire loop 1025, are introduced into the proximal end of the introducer tube 1021 via the cable 10210.

In some embodiments, a tissue segmentation device may utilize one or more wire loops for segmenting tissue specimen and may also be referred to as a loop segmenter. In some cases, a loop segmenter comprising a single wire loop may be utilized. The wire loop may be configured to extend out of a distal end of a lumen (e.g., multi-lumen tube in FIGS. 12A-12C), where the lumen may be shaped and sized to fit within an inner diameter of an introducer tube or trocar. Prior to or during segmentation, the distal wire loop (e.g., wire loop 1025 in FIG. 2, wire loops 11063 in FIG. 12A) may be placed around the tissue. Then, the wire loop may be retracted such that it closes/wraps around the tissue specimen. In some cases, the wire loop may be pulled into the tissue specimen to provide tissue compression. In some embodiments, the lumen may be configured to be lowered (e.g., into the patient incision) such that the tissue specimen contacts the return electrode, which may allow division of the tissue specimen at the location where the active electrode or wire loop contacts the tissue specimen. In some embodiments, the lumen is designed to be reusable. For instance, in some cases, the lumen may be reused multiple times by inserting new segmenting wire(s) prior to use. In some cases, the segmenting wire may be a single use wire. Alternatively, the segmenting wires may be removed after each tissue specimen division and sanitized/cleaned prior to the next use.

Some aspects of this disclosure relate to static or collapsible wire screens. In some embodiments, for instance, when a tissue specimen is pulled into a stationary active electrode, the stationary active electrode may be configured to collapse and retract into the distal end of the actuator instrument. In such cases, the active electrode comprises a deployed position and a stowed position, where in the deployed position the active electrode extends past a distal end of the actuator instrument (or introducer tube) and in the stowed position the active electrode is stowed inside a portion of the actuator. Such a design may facilitate in guiding the stationary active electrode through the patient incision and/or inside a loaded tissue specimen bag. In some embodiments, once the distal end of the actuator or introducer tube is placed into the patient incision, the active electrode is moved from the stowed position to the deployed position. In the deployed position, the active electrode extends or expands from the distal end of the introducer tube, which serves to increase the electrode profile (e.g., active electrode surface area), for instance, for dividing larger tissue specimens.

In one non-limiting example, the active electrode comprises a thin edge that extends from the distal end of the introducer tube (e.g., introducer tube 1021 in FIG. 2). In some aspects, the thin edge protrusion helps limit the exposure size of that active electrode. Alternatively, a substantial portion of the protruding edge of the active electrode may be coated over, thereby leaving all but the most distal edge uncoated, which may also serve to limit the size of the active electrode. In some cases, this active edge protrusion may be part of a retractable feature that allows the electrode edge to be stowed inside the introducer tube, for example, during deployment through the patient incision and, in some cases, the specimen bag. Once in place, the electrode leading edge may be deployed for the division step (i.e., when the tissue is pulled into the electrode edge causing tissue segmentation). It should be noted that, the size and location of this stationary active electrode may determine the shape and location of the tissue division.

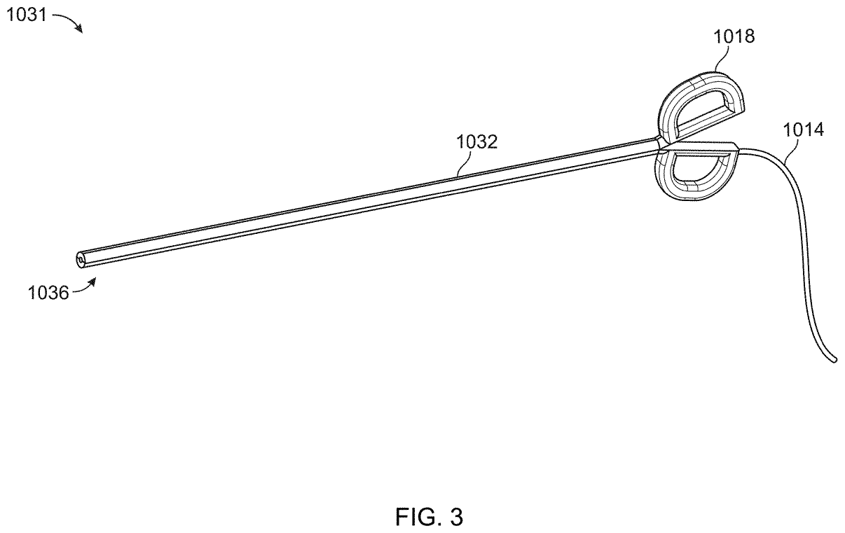

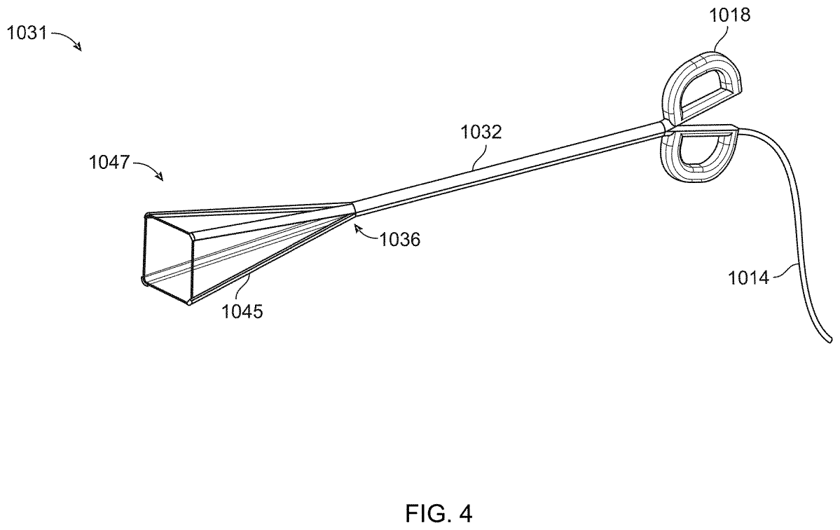

Turning now to FIGS. 3, 4, and 5, which illustrate some examples of a collapsible wire screen electrode, according to various aspects of the disclosure. FIG. 3 illustrates an example of a tissue segmentation device 1031 comprising a tube 1032, one or more handles 1018, and a cable 1014. The tube 1032 may be shaped and sized to house a collapsible wire screen electrode. In some embodiments, the collapsible wire screen electrode is configured to extend from a distal end 1036 of the tube 1032, further described below in relation to FIG. 4.

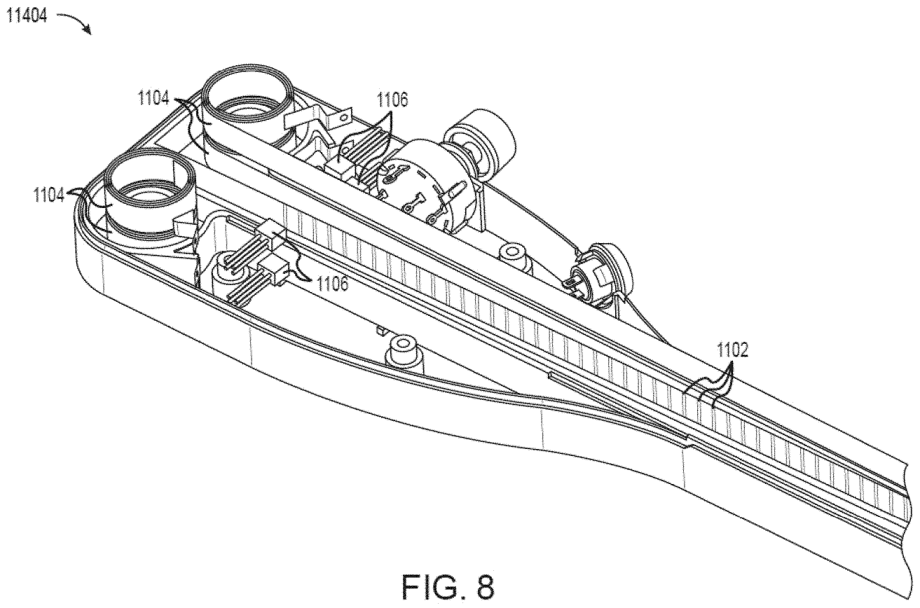

FIG. 4 illustrates the tissue segmentation device 1031 of FIG. 3 in a deployed position, according to various aspects of the disclosure. In this example, the tissue segmentation device 1031 comprises a wire screen electrode 1047 extending from the distal end 1036 of the tube 1032. The wire screen electrode 1047 comprises a plurality of tines 1045 extending from the distal end 1036 of the tube 1032 at an angle (e.g., <90 degrees). In the stowed position (shown in FIG. 3), the plurality of tines 1045 are configured to collapse and/or retract into the distal end of the tube 1032. In some cases, the plurality of tines 1045 are arranged in a parallel or substantially parallel configuration (e.g., inside the tube 1032, or at the distal end 1036 of the tube) when in the stowed position. In some cases, an electrode cable 1014 (e.g., active electrode cable or return electrode cable) may be electrically coupled to the tines 1045 and may be introduced from the proximal end of the tissue segmentation device 1031, for instance, at or near the handles 1018. Further, the tines 1045 (or active electrode wires 1045) may be deployed into the patient incision from the distal end 1036 of the tube 1032. In some cases, the active electrode(s) or tines 1045 may be deployed by a spring-loaded mechanism comprising one or more springs (i.e., to encourage the full deployment of the tines 1045), which allows the active electrode(s) or tines 1045 to expand out of the distal end 1036 of the tube 1032 to receive a larger tissue specimen for segmentation. FIGS. 7, 8. and/or 21 show some non-limiting examples of springs that may be utilized to expand the tines 1045 from the tube 1032, in accordance with one or more implementations. It should be noted that, other applicable mechanisms besides a spring-loaded mechanism are contemplated in different embodiment and the example listed herein is not intended to be limiting.

As most clearly seen in FIG. 4, the active electrode(s) or tines 1045 of the wire screen electrode 1047 may take the shape of a web (e.g., a spider web) when in the deployed position. Such a design may allow the surgeon or user to push the electrode web (or wire screen electrode 1047) into the tissue specimen (not shown) for division.

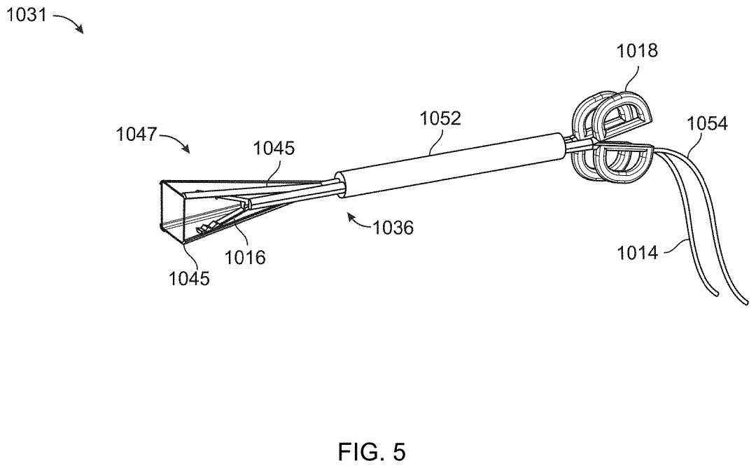

FIG. 5 illustrates the tissue segmentation device 1031 of FIG. 4 including the grasper and a return electrode cable 1014, according to various aspects of the disclosure. The tissue segmentation device 1031 in FIG. 5 implements one or more aspects of the tissue segmentation device(s) described in relation to FIGS. 1-4. In this example, the tissue segmentation device 1031 comprises an introducer tube 1052, a plurality of handles 1018, an active electrode 1054, a return electrode cable 1014, grasping tongs 1016, and a wire screen electrode 1047 comprising a plurality of active electrode(s) or tines 1045. In some cases, the grasping tongs 1016 may comprise a return electrode and/or may be electrically coupled to the return electrode cable 1014. Further, the wire screen electrode 1047 (or electrode web 1047) comprising the plurality of tines 1045 may be electrically coupled to the active electrode 1054. A user or surgeon may individually manipulate the grasper and the wire screen electrode 1047 by way of the handles 1018 provided at the proximal end of the tissue segmentation device 1031. In some cases, the introducer tube 1052 may be similar or substantially similar to the introducer tube 1021 previously described in relation to FIG. 2. Specifically, the introducer tube 1052 may be shaped and sized to fit the grasping tongs 1016 and the wire screen electrode 1047 (i.e., in its stowed or collapsed configuration) within its internal volume. In other words, the grasping tongs 1016 and the wire screen electrode 1047 may be configured to be stowed inside the introducer tube 1052 and expand from the distal end 1036 of the introducer tube prior to (or during) the segmentation procedure.

In some cases, and as seen in FIG. 5, a return electrode grasper (i.e., grasping tongs 1016) may be used to pull the tissue specimen through the expanded wire screen electrode 1047. In some cases, the return electrode grasper may be similar or substantially similar to the return electrode grasper 1011 previously described in relation to FIGS. 1 and/or 2. In some cases, the return electrode grasper comprising the grasping tongs 1016 may be shaped and sized to fit within the introducer or actuator tube 1052. In some embodiments, the expansion method for the mechanism described above may allow the one or more electrodes or tines 1045 to expand/collapse with pressure to adequately match the overall size of the tissue specimen to be segmented.

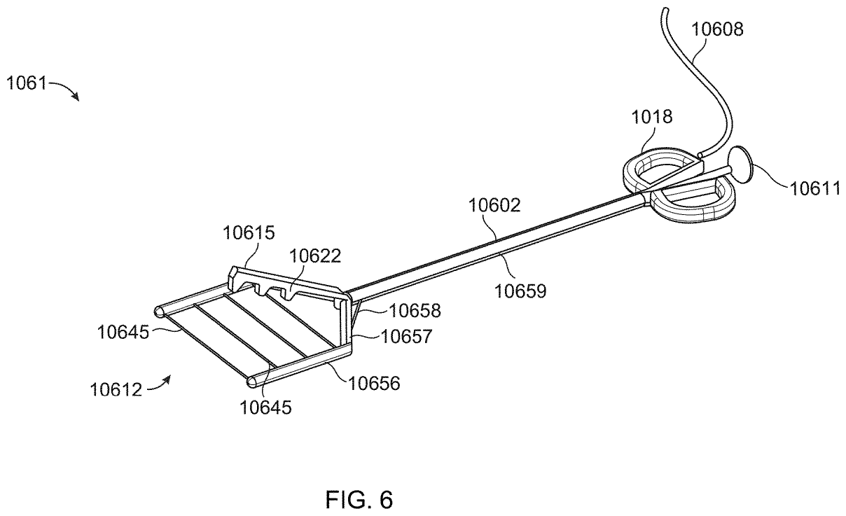

FIG. 6 illustrates another example of a tissue segmentation device 1061, according to various aspects of the disclosure. The tissue segmentation device 1061 implements one or more aspects of the other tissue segmentation devices described herein, including at least tissue segmentation devices 1020 and/or 1031 described in relation to FIGS. 2 and/or 5, respectively. In this example, the tissue segmentation device 1061 comprises a collapsible wire electrode segmenter 10612 positioned at a distal end of a tube 10602, a grasper 10615 positioned at the distal end of the tube 10602, one or more handles 1018 at a proximal end of the tube 10602, a push/pull handle 10611 positioned at the proximal end of the tube, and a cable 10608. In some cases, the wire electrode segmenter 10612 (or simply, wire electrode 10612 or segmenter 10612) comprises a single arm return electrode, for instance, coupled to or part of the grasper 10615.

In some cases, the tube 10602 comprises a non-conductive outer surface and serves as the housing for the one or more electrodes/wires of the tissue segmentation device 1061. One or more components of the tissue segmentation device 1061 may be movable between a stowed/collapsed position and a deployed position. FIG. 6 depicts the tissue segmentation device 1061 in its deployed position, in which the grasper 10615 and the segmenter 10612 protrude/expand from the distal end of the tube 10602. In the stowed position, the grasper 10615 and the wire electrode 10612 are configured to collapse so they can be retracted into the distal end of the tube 10602. The collapsible wire electrode 10612 comprises a plurality of horizontally oriented wires/electrodes 10645 extending between two longitudinal rods 10656. As seen, when in the deployed position, the longitudinal rods 10656 are parallel or substantially parallel to a central axis of the tube 10602. Each of the longitudinal rods 10656 comprises a proximal end and a distal end. Further, each of the longitudinal rods 10656 is coupled to another rod 10657 (or angled rod 10657) at the proximal end, and the proximal end(s) of the angled rods 10657 are coupled to one or more of the grasper 10615 and the tube 10602. For example, the proximal end(s) of the angled rod(s) 10657 may be coupled to a base of the grasper 10615, which is then coupled to the distal end of the tube 10602. Alternatively, the proximal end(s) of angled rod(s) 10657 and the proximal end of the grasper 10615 are coupled to the distal end of the tube 10602.

In some cases, the grasper 10615 comprises the return electrode and is electrically isolated from the active electrodes/wires 10645 of the segmenter 10612. Here, the grasper 10615 comprises one or more teeth 10622 for grasping and/or pulling the tissue specimen (not shown) into position for segmentation. In one non-limiting example, the return electrode may be electrically coupled to the one or more teeth 10622. That is, only a portion of the grasper 10615 may serve as the return electrode. In other cases, a majority or all of the grasper 10615 may form the return electrode. In some cases, one or more of the angled rod(s) 10657 and the longitudinal rod(s) 10656 may be conductive and may form part of the active electrode. Alternatively, the rod(s) and/or the longitudinal rod(s) 10657 may comprise a non-conductive outer surface, in which case the active electrode is formed by the wires 10645. It should be noted that, not all of the wires 10645 may be conductive. In one non-limiting example, every other wire 10645 may have a conductive outer surface. Alternatively, only the first wire (e.g., wire closest to the distal end of the tube 10602) and the last wire (e.g., wire at the distal end of the wire electrode segmenter 10612) may be exposed/have a conductive outer surface.

As noted above, the return electrode may be electrically isolated from the active electrode(s)/wires. That is, the grasper 10615 comprising the return electrode may be electrically isolated from one or more of the wires 10645 and/or the longitudinal rod(s) 10656 during the segmentation procedure. In some cases, the tube 10602 comprises side channels 10659, where each side channel 10659 is shaped and sized to receive a sliding rod 10658 coupled to a corresponding one of the angled rods 10657. The sliding rods 10658 are configured to slide within side channels 10659 based on movement of the push/pull handle 10611. For example, the push/pull handle 10611 may be pulled in the proximal direction to collapse the wire electrode segmenter 10612 and retract the segmenter 10612 and/or the grasper 10615 into the distal end of the tube 10602. Similarly, the tissue segmentation device 1061 may be moved into its deployed position by pushing the push/pull handle 10611 such that the wire electrode 10612 and/or the grasper 10615 extend from the distal end of the tube 10602, as seen in FIG. 6. In some embodiments, the handles 1018 may be used to manipulate the grasper 10615, for instance, to grasp, hold, and/or pull the tissue specimen during the segmentation procedure.

In some cases, the housing or cable 10608 may house one or more electrodes/wires, such as the active electrodes, return electrodes, etc. In some cases, the cable 10608 may also comprise the input power cables used to supply power or energy (e.g., RF energy) to the tissue segmentation device 1061, for instance, via the RF power source 306.

In some other cases, the grasper 10615 and/or the teeth 10622 may be non-conductive. That is, the grasper 10615 may not incorporate the return electrode. In some examples, the tissue segmentation device 1061 comprises one or more collapsible tines (e.g., electrodes/wires 10645), where at least one of the collapsible tines integrates a return electrode connection. In one non-limiting example, one or more of the electrodes/wires 10645 may comprise the return electrode and one or more of the electrodes/wires 10645 may comprise the active electrode, where the active electrode is separate/electrically isolated from the return electrode. For instance, every alternate electrode/wire 10645 may be one of an active electrode and a return electrode. In this way, the plurality of electrodes/wires 10645 integrating the active, return electrodes, and the grasper 10615 may be used to grasp the tissue specimen, for instance, like a carnival grasper machine.

In some cases, the tissue segmentation device 1061 comprises a mechanism for collapsing the expanding tines 10645 and/or for pulling the expanding tines/wires 10645 back into the tube 10602. Further, with the help of RF energy, the tissue specimen may be segmented into horizontal segments as the tines or wires 10645 are collapsed back into the instrument. In some examples, the return electrode may be arranged or incorporated into an opposing force “leg” of the collapsible system, such as, but not limited to, the grasper 10615. The process described above may be repeated to further segment any tissue piece too large for removal from the patient incision.

In some examples, the return electrode is an electrically conducting component that is placed in contact with the tissue specimen. It can either be a component that is located proximal to the bag (e.g., bag 161 in FIG. 16) and wires (e.g., electrodes 153, 155, 157, 159 in FIG. 16) so that when the bag and wires are deployed the return electrode will be located near, or integrated with, the distal end of the device (e.g., electrosurgical instrument 102) and will also be in contact with the tissue specimen. In another embodiment, the return electrode may be coupled to the distal end of the lumen (e.g., multi-lumen tube 11052 in FIG. 12A) in a fixed position. The return electrode (e.g., return electrode 330 in FIG. 15) is electrically isolated from the active wires (e.g., electrodes 322 in FIG. 15, wires 11063 in FIG. 12A) and is of sufficient size to minimize or eliminate cutting and/or reduce heat at the tissue return electrode site. The return electrode may comprise a circular, flat, or rounded disc located near a center of the device distal lumen or can comprise a ring that surrounds the device distal lumen. The return electrode may be applied to the tissue with deployable contact areas located on the distal end of the tissue segmentation device. These contact areas can normally be in a closed position prior to deployment of the bag and upon deployment extend outward beyond the distal end of the device and beyond the diameter of the lumen in a pivoting motion. The resulting geometry has extensions surrounding the distal end opening that form contact points along a circumference in a plane above the distal end of the device lumen. The material of these extensions may be composed primarily of an insulator that can withstand a high temperature with a conductive layer located either in the inner surface and/or the most distal surface of the extension. Alternatively, they may be composed of a metal that is partially coated with an electrically and thermally insulative material such that only the tissue is in contact with the conductive portion of the metal. The tissue segmentation device may be configured so that the active electrodes do not come in contact with the return electrode when the wires have been retracted. For example, the active electrode wires may be channeled away from the return electrode through the use of insulating features attached to or above the return electrode or surrounding the wires, such as, but not limited to, small tubing or tubes (e.g., lumens or channels 11053 in FIG. 12A) that provide electrical insulation and guide the active electrode wires (e.g., wires 11063) and allow them to slide into the tubing during the segmentation procedure, thereby insulating the return electrode from the active electrode wires.

In some embodiments of the present disclosure, an introducer tube (e.g., introducer tube 1021 in FIG. 2, introducer tube 11051 in FIG. 12A) with a distal return electrode may be used in conjunction with the active electrode wire loops (e.g., wire loop 1025 in FIG. 2, wire loops 11063 in FIG. 12A). As noted above, in some cases, the distal return electrode may be incorporated into a grasper or grasping tongs, such as grasping tongs 1016 in FIG. 2, at the distal end of the introducer tube 1021.

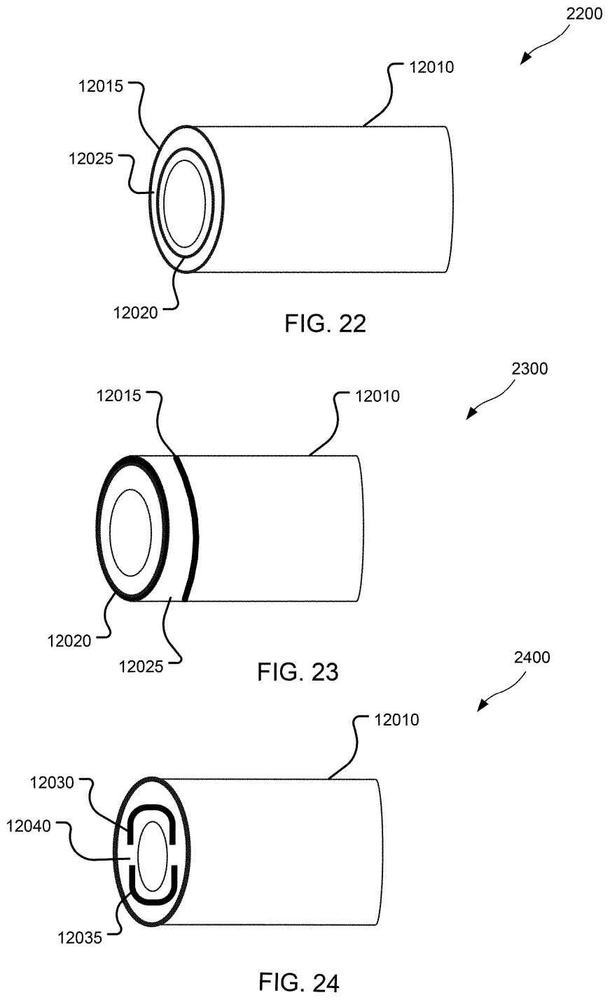

In some other cases, this return electrode may be incorporated into the distal end of the introducer tube 12010 in FIG. 22 (also shown as introducer tube 1021, introducer tube 1052, and/or introducer tube 11051 in FIGS. 2, 5 and/or 12A, respectively), by way of two conducting concentric rings 12015 and 12020. In some embodiments, the insulating ring 12025 (also referred to as space 12025) is positioned between the two concentric rings 12015, 12020 to electrically isolate the two conducting concentric rings 12015, 12020 such that they are not electrically coupled, thereby providing dual electrodes. In some cases, each concentric ring may be electrically coupled to separate sides of a circuit, the circuit configured to measure the impedance or resistance between the two concentric rings 12015, 12020. In some embodiments, an interrogation signal may be applied to the circuit to detect an impedance of tissue in contact across both rings, thus resulting in a first Specimen Contact Quality Monitor (SCQM), where the first SCQM indicates contact (if any) of the tissue with both concentric rings 12015, 12020.

In some other cases, a single conducting ring (e.g., conducting ring 12015 or conducting ring 12020) may be positioned at the distal end of the introducer tube 12010. The conducting ring (e.g., conducting ring 12015) may be electrically coupled to one side of an impedance measurement circuit. In some examples, the other side (or end) of the impedance measurement circuit may be connected to a return electrode (e.g., return electrode 330). In some cases, an interrogation signal may be applied to the impedance measurement circuit to detect tissue impedance between the return electrode and the distal end of the introducer tube, thus resulting in a second Specimen Contact Quality Monitor (SCQM). In some examples, this second SCQM is configured to detect (1) contact of the tissue specimen with the return electrode, and (2) contact of the distal end of the introducer tube with the tissue specimen.

In some aspects, the concentric ring design discussed above may help determine (e.g., before RF energy is delivered) if the tissue is in contact with the introducer tube (e.g., shown as introducer tube 1021, 1052, 11051 in FIGS. 2, 5, 12A, respectively).

FIG. 22 depicts an embodiment (2200) showing concentric rings 12015, 12020 positioned at a distal end of an introducer tube 12010, where the concentric rings 12015, 12020 are positioned in a planer orientation on the same distal surface of the introducer tube, according to various aspects of the disclosure. Furthermore, FIG. 23 depicts an alternate embodiment (2300) where a single electrode, such as conductive ring 12020, is positioned on a distal end of an introducer tube 12010 and a concentric ring 12015 is positioned on the side surface of the introducer tube 12010. In some cases, the rings 12015, 12020 are separated by an insulating ring or space 12025, the insulating ring 12025 positioned on the side surface of the introducer tube 12010. In some circumstances, the embodiment (2300) shown in FIG. 23 allows larger separation of the concentric rings 12015, 12020 while also allowing a larger surface area of each concentric ring.

In some other cases, two electrically isolated conducting hemispheres (e.g., shown as conducting hemispheres 12030 and 12035 in an embodiment 2400 depicted in FIG. 24) are arranged and positioned on the distal end of the introducer tube 12010. In some examples, the conducting hemispheres 12030 and 12035 are electrically isolated by an insulating gap 12040 positioned between the two conductive hemispheres. The insulating gap 12040 helps ensure that the conducting hemispheres 12030, 12035 are not electrically coupled, thereby providing a dual electrode configuration at the distal end of the introducer tube 12010. Other types of dual electrode configurations may be implemented in different embodiments, and the examples listed above are not intended to be limiting. For example, in some cases, the return electrode may be electrically coupled to one or more of the concentric rings (e.g., concentric rings 12015, 12020). Connection of the return electrode to a single ring or conducting hemisphere may help provide the return connection of RF current through the connected concentric ring. In such cases, the other concentric ring (i.e., not connected to the return electrode) serves as the interrogation or tissue sensing ring. Alternatively, in some embodiments, a SCQM may be implemented by electrically connecting a return electrode to both concentric rings (or conducting hemispheres), i.e., a dual electrode design. In such cases, the return current is shared by both rings/conducting hemispheres coupled to the return electrode.

In some circumstances, the return electrode(s) may need to be protected from the wires (e.g., active wires/electrodes) traveling through the lumen, such as the multi-lumen tube described in relation to FIGS. 12A-12C, to avoid shorting. In some instances, this protection may be achieved by designing a slight shoulder in the inside surface of the lumen, where the slight shoulder protrudes distal to the lumen, thereby providing a path for the active electrode wire(s) to travel around the return electrode without contacting the return electrode. In some other cases, the isolated concentric rings may be placed on the outer surface of the lumen, for example, at or near the distal end of the lumen or introducer tube (e.g., as shown in FIG. 23). In either of these cases, the return may rely on the contact between the tissue and one or more of the return electrodes pairs to satisfy the SCQM.

In some cases, the dual return electrode configuration (e.g., implemented using conducting concentric rings 12015, 12020 separated by an insulating ring 12025) described above can also be used to provide a tissue contact indicator. In some circumstances, this configuration may be used as a monitoring circuit, for instance, to identify if the introducer tube has been lifted when RF activation is requested. Similar in line with the SCQM described above, in some embodiments, this monitoring circuit may be configured to provide an interrogation signal between the dual electrodes. Alternatively, one of the electrodes of this monitoring circuit may be utilized to monitor the tissue resistance, while the other electrode may be utilized to monitor specimen contact quality.

In some circumstances, for instance, for small incision procedures, a portion of the containment bag assembly may be kept outside of the patient incision to minimize the volume of product (e.g., tissue specimen, connection point of the electrode wires to the actuator, etc.) that passes through the patient incision. In one non-limiting example, an integrated actuator/containment bag system may be provided, which serves to minimize the extra volume needed to accommodate the segmentation instrument(s). In one non-limiting example, the integrated actuator/containment bag system may be configured to remove any wire electrode connection junction which adds extra volume.

In some other cases, the electrode wire/actuator connection point (e.g., connection point between electrode wires 322 and actuator 304 in FIG. 15) may be moved to a location that is outside of the patient incision, as shown in FIG. 15. In some embodiments, elongated electrode wires or wire loops, such as the ones described in relation to FIGS. 12A-12C, 15, and/or 16, may be utilized. These elongated electrode wires, formatted as wire loops (e.g., wire loops 11063, wire loops 322, wire loops 153, 157, 159, etc.) may be shaped and sized to pass through a small diameter, flexible or rigid, single, or multi-lumen tube (e.g., multi-lumen tube 11052), where the lumen tube may be used to pass the electrode wires through the patient incision. In some embodiments, for instance, for a multi-lumen tube, a high temperature cap or ring may be used at the distal end of the tube (e.g., multi-lumen tube 11052 in FIG. 12A) as a proxy for the introducer tube 11051 used to protect the patient incision.

As seen, FIG. 12A illustrates an example of a process flow 1101-a, according to various aspects of the disclosure. In some cases, the process flow 1101-a is directed to using a tissue segmentation system, wherein an insertion tube is inserted into a patient incision, and comprises: (1) providing an insertion tube or introducer tube 11051, (2) providing a multi-lumen tube 11052 having a plurality of lumens or channels 11053, (3) providing a lubricious connector 11062 having a plurality of pins 11061, (4) receiving the plurality of pins 11061 in the plurality of lumens or channels 11053 of the multi-lumen tube 11052, as shown at the end of step A, and (5) extending the insertion/introducer tube 11051 in a downward direction, as shown at the end of step B.

In some cases, the pins 11061 may be shaped, sized, and/or positioned to be received in the lumens/channels of the tube 11052. Further, the multi-lumen tube 11052 is shaped and sized to fit within an inner diameter of the introducer tube 11051. In some cases, the plurality of pins 11061 on the proximal portion of the connector 11062 are coupled to the plurality of segmenting wire loops 11063 shown at the distal portion of the connector 11062. The connector 11062 may have a plurality of through holes or other applicable features to enable the connection between the segmenting wire loops 11063 and the pins 11061. In some examples, the connector 11062 (also referred to as a lubricious connector 11062) is configured to reduce or minimize friction between the multi-lumen tube 11052 and the segmenting wire loops 11063.

The illustration on the left of the page in FIG. 12A depicts the multi-lumen tube and the connector prior to the connection. At step A, the connector and pins are connected to the multi-lumen tube 11052, in which case the pins are received in the lumens 11053 of the tube 11052 and the connector 11062 is positioned at a distal end of the multi-lumen tube. In some examples, at step B, the insertion tube 11051 is extended in the distal direction (i.e., down in the page) such that the distal end of the insertion tube 11051 extends past the distal end of the connector 11062 and/or at least a portion of the insertion tube 11051 surrounds the segmenting wire loops 11063 extending distally from the connector 11062. In other words, the insertion tube 11051 is extended (or pushed down) such that at least a portion of the insertion tube (e.g., the distal end of the insertion tube) is inserted into the patient incision.

FIG. 12B illustrates an example of a process flow 1101-b, according to various aspects of the disclosure. In some cases, the process flow 1101-b is directed to using a tissue segmentation system, wherein a multi-lumen tube is inserted into a patient incision, and comprises: (1) providing an insertion tube or introducer tube 11051, (2) providing a multi-lumen tube 11052 having a plurality of lumens or channels 11053, (3) providing a lubricious connector 11062 having a plurality of pins 11061, (4) receiving the plurality of pins 11061 in the plurality of lumens or channels 11053 of the multi-lumen tube 11052, as shown at the end of step A, and (5) inserting the multi-lumen tube 11052 into the patient incision by pushing it distally (i.e., down in the page), as shown at the end of step B.

In yet other cases, for instance, for a flexible multi-lumen tube (e.g., multi-lumen tube 11052 in FIG. 12C), a trocar can be used to provide the required stiffness needed for the lumen tube. In some embodiments, such as, when a flexible multi-lumen tube is used, a stiffening rod (e.g., shown as stiffening rod 11099 in FIG. 12C) may be added to at least one of the channels 11053. The stiffening rod 11099 may be added to the lumen channel prior to, or during, the use of the actuator. In some circumstances, this stiffening rod 11099 may help provide a stiff counterforce for wire loop segmentation through the tissue specimen.

FIG. 12C illustrates an example of a process flow 1101-c, according to various aspects of the disclosure. In some cases, the process flow 1101-c is directed to using a tissue segmentation system, wherein a stiffening rod is utilized to provide additional stiffness or support for a flexible multi-lumen tube, and comprises: (1) providing an insertion tube or introducer tube 11051, (2) providing a multi-lumen tube 11052 having a plurality of lumens or channels 11053, (3) providing a lubricious connector 11062 having a plurality of pins 11061, (4) providing a stiffening rod 11099, (5) inserting the stiffening rod 11099 through a proximal portion of the multi-lumen tube 11052 and into one of the lumens (e.g., a central lumen) of the multi-lumen tube, (6) receiving the plurality of pins 11061 in the plurality of lumens or channels 11053 of the multi-lumen tube 11052, as shown at the end of step A, and (7) pushing the rod 11099 in a distal direction (i.e., down in the page) such that it passes through all or a majority of the length of the corresponding lumen/channel, as shown at the end of step A. In some cases, the rod 11099 may be of sufficient length that it extends from the distal end of the multi-lumen tube 11052 and into the connector 11062, as shown in the illustration on the right of the page in FIG. 12C. In such cases, the connector 11062 may have an additional receiving hole (i.e., in addition to the holes for holding the pins 11061) that is shaped and sized to receive the rod 11099.

In some other cases, connectors (e.g., connector housing and connector pin assembly described in relation to FIG. 11A) having a smaller cross-section footprint as compared to the prior art may be utilized, which may allow the connection to remain with the portion of the containment bag that enters through the patient incision. In some cases, stackable connectors (e.g., similar to those found in a rifle ammunition clip) may be utilized. In some other cases, connectors may be temporarily attached end-to-end, which may help in minimizing the cross-sectional shape area, while also allowing the length to grow for a plurality of connectors.

In many laparoscopic procedures a surgeon may wish to place surgical instruments (e.g., segmenting wires, grasper, scissors, etc.) through a patient incision using a trocar. In some circumstances, trocars allow for a tight pneumatic seal of the patient incision while surgical instruments are passed freely through the trocar central shaft. In some cases, trocars also comprise an auxiliary port to allow for patient cavity insufflation using carbon dioxide (CO₂) gas. In some embodiments, a trocar comprising an atraumatic distal surface (e.g., a blunt trocar) may be used during tissue segmentation. In some cases, a sharp insert may be placed at or near a distal end of the trocar to aid in placement. Further, this sharp insert may be removed after placement (e.g., in the peritoneum). In some cases, the trocar may be shaped and sized to pass one or more laparoscopic surgical devices, such as, but not limited to, a grasper, segmenting wires or wire loops, a collapsible wire screen electrode, etc. In some cases, the deployment instrument (e.g., deployment instrument 1004 in FIG. 13) may be inserted through the trocar, for instance, when segmentation is needed.

In some embodiments, the specimen/containment bag (or simply, bag) used with the system of the present disclosure may contain one or more wires, where the one or more wires extend through a small lumen or lumen channels of a multi-lumen tube, as shown in FIGS. 12A-12C. In some embodiments, the lumen tube (e.g., multi-lumen tube 11052) may have a smaller diameter than the inner diameter of the trocar or introducer tube 11051. In some cases, the multi-lumen tube 11052 may be shaped, sized, and/or configured to extend from the top of the specimen bag and into the introducer tube 11051. In some cases, a connecter (e.g., lubricious connector 11062) may be positioned and arranged near a distal end of the multi-lumen tube 11502, either as a separate connector or integrated into its distal end. In some embodiments, after the tissue is loaded, the bag (not shown in FIGS. 12A-12C but shown as bag 10101 in FIG. 11A) may be released from the spring arms and may be pulled up around the multi-lumen tube 11502 and out of the trocar/introducer tube 11051 for exteriorization. Further, with the bag exteriorized, the deployment instrument (e.g., deployment instrument 1004 in FIG. 13) may be removed, thereby exposing the lumen tube 11052 and/or connector 11062. Following removal of the deployment instrument (e.g., deployment instrument 1004 in FIG. 13), a tissue segmentation device (e.g., segmenting wires 11063, tissue segmentation device 1020, tissue segmentation device 1031, tissue segmentation device 1061, etc.) may be connected to the connector 11062. In such cases, the lumen or multi-lumen tube 11502 may help provide a counter force, where the counter force facilitates tissue segmentation. After the tissue segmentation is complete, the lumen may be removed along with the segmentation instrument (or tissue segmentation device) to facilitate tissue removal from the specimen bag, patient incision, etc. In an alternate embodiment, the trocar or introducer tube 11051 may be removed (e.g., by lifting it up and out of the patient incision), thereby leaving the lumen and/or exteriorized bag opening extending from the incision site.

In some examples, tissue segmentation devices (e.g., employing RF energy for the segmentation procedure) may be adapted to create a reusable portion that works with a disposable portion of the segmentation instrument, further described in relation to FIGS. 9-10C and 18-19. In some aspects, such a design helps reduce overall procedure cost and/or the amount of disposed material or waste created with each use. In some embodiments, a tissue segmentation device or segmentation instrument may comprise a reusable portion and a disposable portion, further described in relation to FIGS. 9-10C and 18-19. In some cases, this reusable segmentation instrument may comprise a tensioning mechanism, where the tensioning mechanism utilizes a motor to apply a force to advance/retract the segmenting wires. In one non-limiting example, a direct current or DC motor may be utilized in the tensioning mechanism. Using a motor, such as a DC motor, may help advance or retract the position of the segmentation instrument's tensioning mechanism automatically (i.e., with minimal user adjustment). This allows easy reloading of the segmentation instrument to prepare for the next use, as compared to the prior art. Additionally, or alternatively, the DC motor can be incorporated with an encoder to determine real time position information of the wire travel, for instance, during cutting and/or reloading as the segmentation instrument is prepared for the next use. In some examples, the use of the DC motor in the tensioning mechanism may also allow the segmenting wires to be automatically tensioned (e.g., for cutting). Furthermore, the DC motor may help revert the tensioning mechanism to the pre-load position after the segmentation is complete. In some embodiments, the reusable portion of the tissue segmentation device may include the electronics required for communications with one or more of a controller, the tensioning mechanism, and the user controls. In some other cases, the reusable portion of the tissue segmentation device may include the controller, where the controller may be configured to control the operations of the DC motor and/or the tensioning mechanism. In some embodiments, the disposable portion of the tissue segmentation device may be limited to the interface of the tissue segmentation device with the segmenting wires. In some examples, the features and embodiments described above can be used on their own or in conjunction with and as improvements to the systems described below. According to aspects of this disclosure, a reusable DC motor or actuator may be utilized to apply a tensioning force to one or more active electrode wire loops. Described below are some non-limiting examples for achieving the detachable connection of the reusable DC motor to the active electrode wire loop, in accordance with one or more implementations.

It should be noted that, in some embodiments, the segmentation instrument comprising the tensioning mechanism and DC motor may be incorporated in an entirely disposable system. That is, the disclosure of a segmentation instrument comprising a first reusable portion and a second disposable portion is not intended to be limiting.

FIG. 9 illustrates an example of a reusable portion 1071 of a tissue segmentation device, according to various aspects of the disclosure. Specifically, FIG. 9 depicts the detachable connection of a reusable DC motor with a disposable portion of a segmentation instrument or tissue segmentation device. In some cases, the reusable portion 1071 comprises a DC motor 10712 having a paddle 10714. FIG. 9 also illustrates the disposable portion 10711 of the segmentation instrument. In this example, the disposable portion 10711 comprises a plurality of wire loop spools 10718. In one non-limiting example, each active electrode wire loop may be affixed to a wire loop spool 10718 for the disposable portion of the segmentation instrument. In some examples, the wire loop (not shown) may be connected directly, or alternatively, with an intermediate conductive cable (e.g., cable 10832 in FIGS. 10A and 10B) to the DC motor 10712. Further, each wire loop spool 10718 may comprise a central slot 10728, where the central slot 10728 is shaped and sized to fit the rotating paddle 10714 of a corresponding DC motor 10712. In some embodiments, the reusable DC motor 10712 may temporarily latch with the disposable portion 10711 of the actuator, which may serve to aid in the alignment of the DC motor paddles 10714 with the central slots 10728 of the wire loop spools 10718. In some instances, the DC motors 10712 may be driven to ‘start’ and ‘end’ at this ‘in line’ alignment (i.e., when the central slot 10728 is aligned with the channel 10720 of the disposable portion), for instance, before the connection(s) are made. With the slotted spools 10718 held in place at initial position, the one or more DC motor 10712 may be slid (as depicted by arrow 10716) to engage the respective mating spools 10718. In some cases, a feature or mechanism (e.g., a living hinge snap feature between the disposable portion 10711 and the reusable DC motor 10712) can be provided to help hold the DC motor in position to maintain engagement with the disposable portion 10711 of the actuator containing the plurality of wire loop spools 10718.

Some aspects of the present disclosure relate to providing a connection of RF energy from a DC motor to an electrode wire loop, for instance, for an actuator or segmentation instrument comprising one or more reusable components. FIGS. 10A and 10B illustrate an example of an electrical connection mechanism for coupling a DC motor to an electrode wire loop spool, according to various aspects of the present disclosure. FIG. 10A illustrates a perspective view of an electrical connection mechanism 1081-a and FIG. 10B illustrates an exploded view of an electrical connection mechanism 1081-b. In some examples, the electrical connection mechanism 1081-b may be similar or substantially similar to the electrical connection mechanism 1081-a in FIG. 10A.

As seen, FIG. 10A depicts a wire loop spool 10718 comprising a central slot 10728, where the wire loop spool 10718 and the central slot 10728 are similar or substantially similar to the ones previously described in relation to FIG. 9. In some cases, the central slot 10728 of the wire loop spool 10718 is shaped and sized to receive a rotating paddle (e.g., paddle 10714 in FIG. 9) of a DC motor. In some embodiments, the active electrode loop may be electrically connected to a conductive cable or strand 10832, where the strand 10832 may be a single wire made of a conductive material (e.g., copper, silver, or another metal). Alternatively, the cable or strand may comprise an insulative or non-conductive outer surface (e.g., rubber, polymer) surrounding a conductive wire or filament. In yet other cases, the strand 10832 may have a conductive coating (i.e., on the outer surface) surrounding a non-insulative material. In either case, at least a portion of the strand 10832 is conductive. In some examples, the cable or strand 10832 is attached to a connecting element 10831, where the connecting element 10831 is shaped and sized to fit into a slot 10841 (shown in FIG. 10B) in the wire loop spool 10718. Once installed, the top portion of the connecting element 10831 may be bent (also shown in FIG. 10B) to keep the connecting element 10831 firmly lodged within the wire loop spool. In some embodiments, a conductive metal disk 10833 comprising a central hole 10835 may be adhered or affixed to a top surface of the wire loop spool 10718. The central hole 10835 may be shaped and sized to receive the central slot 10728 of the wire loop spool. In some aspects, this disk 10833 helps provide a static and/or consistent conductive path as the wire loop spool 10718 is rotated during actuation. In some embodiments, this conductive disk 10833 may be configured to mate with a drag strip connection 10834, for instance, to supply a RF energy signal from the DC motor to the active electrodes/wires.

In some other cases, a spring-loaded contact using a plunger may be utilized. For instance, the spring-loaded contact may employ a plunger, where the plunger is configured to remain in contact (e.g., with the wire loop spool) during rotation, thereby helping ensure continuous RF conductivity with the wire loop spool. In order to minimize mechanical drag forces in the segmentation system, a bearing assembly comprising multiple ball bearings may be utilized, where the bearing assembly is configured to contact the ring. The metal subcomponents of the bearing assembly may also serve to ensure continuous RF conductivity through the bearing assembly and to the segmenting wires wound around the wire loop spool. In this example, the bearing assembly comprising the multiple ball bearings may assist in reducing the frictional drag experienced by the wire loop spool while enabling the connection (e.g., with the wire loop spool) to be maintained during rotation.

### Independent Pretension

In some embodiments, a separate means to pre-tension the tissue sample may be provided by way of an insulative layer between the wire electrode and the tissue. This layer may be a pressurized air layer, a non-conductive fluid layer, or an insulating film or layer applied between the wire and tissue, which may serve the alternative function of applying the tension to the tissue sample. Alternatively, the insulative layer could be achieved with the design of the bag, the wire attachment, and the pre-tension mechanism such that a gap results in the tissue wire/bag interface during operation. In some cases, power or RF energy may be applied to the desired wire set to be activated. Further, after sufficient power having a voltage is applied, the wire set may be pulled to the surface of the tissue to begin the cutting effect. Alternatively, after RF energy or power is applied to the electrode/wire set, the wire set may mechanically or electrically (e.g., due to a rise in temperature) break through the separation layer and begin the cutting effect. Generally stated, any easily electrically removable (or degradable) adhesive or retaining volume to hold the wire electrode in place may be provided. In some cases, when current is passed through the bare wire electrode it generates a heating effect, where the heating effect is based at least in part on the magnitude of the current and the resistance of the bare wire electrode. Upon electrical input, the bare wire electrode breaks through the retaining medium (adhesive/retaining volume) or film as a result of the heating effect at the bare wire electrode. This easy to degrade medium or film may also provide a pseudo air-gap, to promote initiation of the tissue cutting effect. In some embodiments, the degradable medium or film may have a melting point that is above typical room temperature (e.g., >25 degrees C.) to prevent the degradable medium or film from melting when stored under general operating conditions. However, the degradable medium or film may be configured to degrade when a current that is at or above a threshold is passed through the bare wire electrode.

In some embodiments, after the tissue specimen is loaded into the specimen bag and before RF energy is applied, the electrode/wire assembly may be pretensioned in order to secure the tissue specimen with respect to the wires. This wire pretension also helps embed the wires into the specimen prior to the application of RF energy—thus minimizing the potential spread of elevated temperatures outside of the intended specimen. This wire pre-tensioning can be accomplished with an independent mechanism or combined with the mechanism used for mechanical tension during the specimen cutting process. Pretension values may need to stay below the ultimate tensile value of the wires to which the pretension mechanism is attached. Ideal pretension values occur in a range that mechanically embeds the wires in the tissue specimen (i.e., prior to cutting) and balances the progression of the wire movement through the specimen while getting the optimal cutting effect (i.e., temperature rise in the areas surrounding the specimen are below a threshold) from the RF energy. In one non-limiting example, this pretension may be in the range of 40-100 psi for each electrode/wire. In some cases, this pretension range may be lower than 40 psi if other means are used to secure the specimen.

In some embodiments, for example, for a reusable system where one or more DC motors are used, the pretension step may be done manually and prior to the connection of the one or more DC motors. In one embodiment, the slotted spools (e.g., wire loop spools 10718) previously described in relation to FIGS. 9, 10A, and/or 10B may comprise one or more features on the spool that are shaped and configured to interact with the DC motor housing to prevent the spool from rotating backwards. In other words, the spools 10718 may only be allowed to rotate in a direction that causes the connected wire loops to retract. With this arrangement, each wire loop spool 10718 may be manually or mechanically wound to pretension each wire loop, where the pretensioning may be performed in advance of the DC motor connection. After winding, each wire loop may be configured to remain in its pretensioned state. In some embodiments, the DC motor system (e.g., DC motor 10712 in FIG. 9) may then be connected, for instance, for tensioning and retracting the wire loops during tissue division. Described below are some non-limiting examples for winding wire loop spools for pretensioning, in accordance with one or more implementations.

In one embodiment, the exposed portion of the wire loop spool (e.g., wire loop spool 10718) may comprise a feature that may be grabbed and/or twisted by the user (e.g., a surgeon) to wind the wire loop spool. In one non-limiting example, a key, a rod, or another similar item, may be inserted into a receiving hole or slot in the wire loop spool to manually wind said wire loop spool (e.g., like a winding clock). In yet other cases, the central slot 10728 of the wire loop spool 10718 may be manually rotated to wind said wire loop spool 10718. In another embodiment, the wire loop spool may comprise a cable or a constant torque spring (e.g., constant torque spring 1091 in FIG. 10C) that can be wound around the wire loop spool and pulled to twist each wire loop spool for pretension. In some cases, the cable-like feature, or other mechanical engagement features, may be shaped and sized such that a portion of the pretension mechanism breaks away (or disengages) after a threshold amount of force or torque is applied. In some instances, this threshold may be high enough to ensure that each of the wire loops are sufficiently pretensioned, yet small enough that it is below the mechanical tensile strength of the wire loop.

In another example, a method for creating independent pretensioning of the wire loop spools is provided. In some cases, each motor (e.g., DC motor) may be driven to a preset tensioning force. Further, the motor may be held in this position (e.g., at the preset tensioning force) until the segmenting wire starts slicing the tissue specimen, at which point the tensioning force applied by the motor may be modified (e.g., increased) to perform the segmentation.

Reusable Motor Drive for Use with a Plurality of Electrode Wires

For an actuator system (or segmentation instrument) utilizing a plurality of electrode wires, the discussion above outlines various techniques to individually pretension each electrode wire. In some embodiments, each wire loop spool is individually coupled to a motor, such as a DC motor, which allows the pretensioning force for each wire loop spool or segmenting wire to be individually set. In some other cases, the plurality of wire loop spools may be coupled to a single DC motor, where the DC motor may be individually coupled to each of the plurality of electrode wire tensioning mechanisms or wire loop spools. In yet other cases, the plurality of wire loop spools may be split up into groups (e.g., 2 or 3 wire loop spools per group) and each group may be coupled to a different DC motor.

Additionally, or alternatively, a cam or belt system may be utilized, which may serve to further decrease manufacturing costs and/or minimize user interaction. In this example, a single DC motor may be selectively linked to each electrode wire tensioning mechanism (e.g., wire loop spool, or rack), which may allow the use of one tension drive motor (e.g., DC motor) with limited user interaction.

### Velocity and Torque Parallel Controls

In some embodiments, a variable force mechanism may be utilized to pretension the segmenting wires (i.e., prior to segmentation) and/or apply the tensioning force (i.e., during segmentation). In some cases, the variable force mechanism may be used in addition to, or in lieu of, the constant force tensioning mechanism described above. In some cases, the variable force mechanism is configured to apply the load (or pulling force) to the segmentation wires, where the load may be varied during the course of the segmentation procedure. For example, the load or pulling force may be varied during the cut from a high value to a lower value, or alternatively, from a low value to a higher value. In some circumstances, such a design helps keep the impedance more consistent as the segmenting wires encounter variances in tissue parameters (e.g., cross-sectional size and other applicable parameters or properties of the tissue specimen), which helps enhance the quality of the cut.

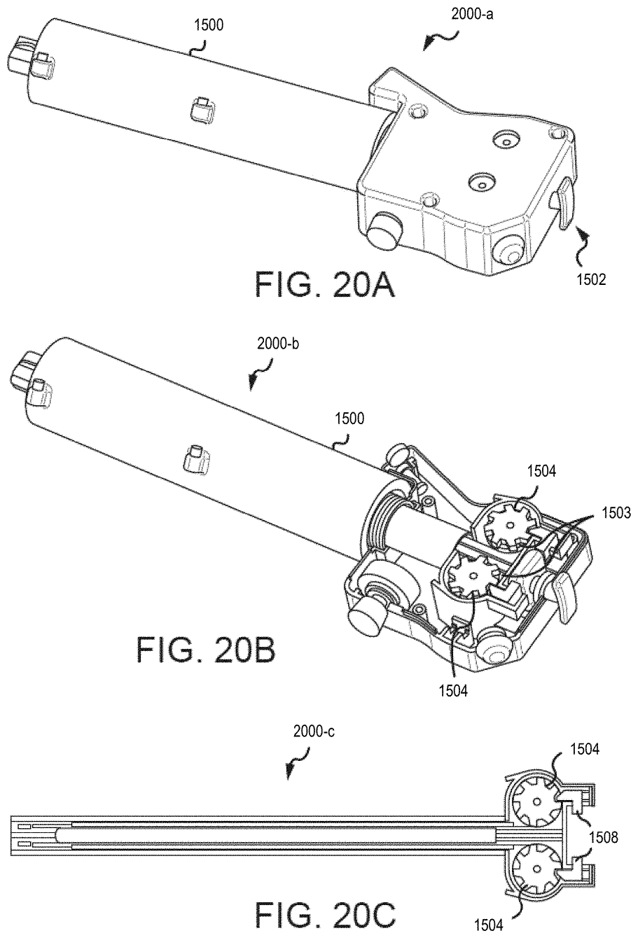

In some cases, the variable force can be applied in a linear reduction using a starting applied force and a predetermined finishing force that would be chosen to model typical tissue compression and sizes. It can also be an exponential decay that models the increase in force as the wire shape changes. In some embodiments, the variable force applied to the tensioning mechanism may be delivered with a DC motor. This motor may be coupled to a segmenting wire with a spool, such as a winch or a worm gear, as shown in FIGS. 20A-20C. In other cases, the motor may be coupled to the segmenting wire with a rack and pinion that travels a length that is at least the total wire cutting length required for cutting the largest specimen (i.e., the largest specimen being cut during said segmentation procedure). In some cases, the DC motor may be used with a current driver, where the current driver is configured to modulate the applied force based on the measured tissue impedance. In this manner, the variable force mechanism applies the maximum force to the wire that also maintains the ability of the generator (e.g., RF generator) to deliver power to the tissue. The DC motor may also be selected with an intrinsic load characteristic (e.g., torque-current curve) that is in line with the range of applied forces desired to allow the force delivered by the DC motor to be controlled with a constant current. For example, a DC motor having a specific torque-current curve may be selected such that, when used with the tensioning mechanism coupled to the segmenting wires, it is configured to apply a force (within a range), where the force can be controlled using a constant current. In other words, the selected DC motor is configured to deliver a force when controlled by a constant current, where the delivered force stays within a desired range (e.g., between an upper and a lower threshold) when the DC motor is used with the tensioning mechanism and the segmenting wires, based at least in part on the torque-current curve of the selected DC motor.

In addition to using a DC motor as a variable force mechanism, in some circumstances, segmentation can be further enhanced by controlling the velocity of the segmenting or cutting wires. In some cases, the velocity of segmenting wire(s) may be controlled by controlling the velocity of the DC motor. In some embodiments of the present disclosure, the velocity of the motor velocity and/or the cutting wires may be controlled using motion feedback, for instance, through the use of a rotary encoder. Alternatively, the voltage used to drive the motor may be adjusted using pulse width modulation (PWM). In some cases, PWM of the voltage drive coupled to the DC motor may help control the motor and/or cutting wire velocity. In some other cases, the force applied by the variable force mechanism can also be controlled by monitoring the average current delivered to the DC motor. In some circumstances, increasing the duty cycle of the PWM may increase the velocity of the motor (e.g., given that the maximum drive force of the DC motor is not exceeded, otherwise the DC motor may stall, which rapidly increases the current through the DC motor). In some cases, the velocity of the motor may need to be controlled to ensure the maximum drive of the DC motor is below a threshold, for instance, by using a maximum setpoint of applied force (or torque). In some aspects, this creates a control system that (1) helps segment the tissue specimen at a self-regulating velocity and/or (2) maintains an applied force that is less than the maximum force setpoint. As the tissue impedance is a function of the force applied by the segmenting wire on the tissue, in some cases, the velocity setpoint can also be simultaneously adapted (e.g., adjusted in real time) to result in a constant or substantially constant tissue impedance during the segmentation procedure.

### Motion Sensing

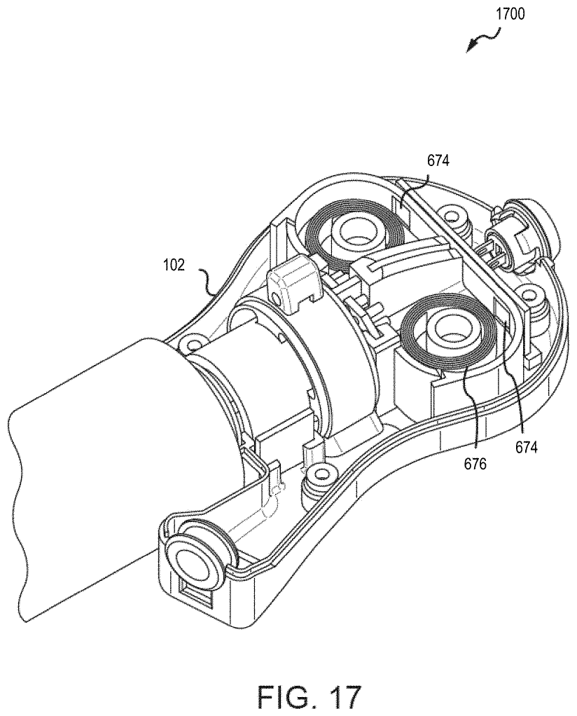

FIG. 17 illustrates an example of a sensing device 1700 configured for use in a tissue segmentation device, according to various aspects of the disclosure In some embodiments, an analog optical reflective sensor (e.g., optical reflective sensor 674 in FIG. 17) may be provided to determine a linear travel distance of the tensioning mechanism (e.g., a spring or another force application mechanism, such as spring 676 in FIG. 17). In some cases, the analog optical reflective sensor 674 may be positioned in close proximity to each spring or force application mechanism 676 of the segmentation instrument. Further, the optical reflective sensor 674 may be focused on a location of the spring 676 such that as the spring 676 recoils, the optical reflective sensor 674 is configured to measure the proximity of the spring 676 relative to the optical reflective sensor 674. This proximity can then be used to infer the linear distance of travel of the spring or force application mechanism 676. In some cases, this linear travel distance may also be used to calculate an average velocity of travel, for instance, based on measuring the time taken to traverse said linear travel distance.

In some embodiments, an analog hall effect sensor may be used in lieu of the optical reflective sensor. In some cases, the optical reflective sensor described in relation to FIG. 17 may be replaced with an analog hall effect sensor, in accordance with one or more implementations. Similar to the optical reflective sensor, the analog hall effect sensor may be positioned in close proximity to each spring or force application mechanism 676. Further, the spring 676 may be manufactured of a ferromagnetic material, which allows it to be magnetized in its coiled position along the axis of linear travel manor in which it recoils. When the spring is tensioned using the tensioning mechanism, it comprises a pattern of North and South poles along its axis. In some cases, the hall effect sensor may be positioned at or near the spring 676. Further, the hall effect sensor may be focused on a predefined location on the spring such that as the spring recoils, the hall sensor outputs a sine wave for each rotation of the spring coil radius. The number of rotations of the coil may be used to infer the linear distance of travel. Similar to the optical reflective sensor, the linear distance of travel may also be used to estimate an average velocity of travel. In some embodiments, the spring 676 may not be composed of a ferromagnetic material and may instead have a permanent magnet mounted to its inner radius, which serves to achieve the same or similar effect.

### Reusable Wind-Up Clock Springs

In some embodiments, a tensioning mechanism may include a constant force spring (e.g., shown as constant force spring 1091 in FIG. 10C) and/or other mechanisms such as a pulley system (e.g., shown as pulley 10944 in FIG. 10C), a cable drive or winch system, non-linear springs, linear drive with rotational coupling such as gears or contact coupling, linear drive with magnetic coupling, linear drive with manual control, and/or, as previously described, an electromechanical drive, such as a servo or stepper motor drive or linear actuator.

According to aspects of this disclosure, a reusable wind-up clock spring may be utilized, for instance, for retraction of the wire from the wire loop spool. In some embodiments, a spring mechanism configured to produce a constant (or substantially constant) torque may be used when a wire loop spool (e.g., wire loop spool 10718 in FIGS. 9, 10A, and/or 10B) is integrated into the tensioning mechanism.

FIG. 10C illustrates an example of a constant torque spring 1091 configured to produce a constant torque for retracting a cable 10946, according to various aspects of the disclosure. In some examples, the cable 10946 may be similar or substantially similar to the cable 10832 (i.e., described in relation to FIG. 10A) and may be coupled to the segmenting wire loop (not shown) being retracted. In some cases, the constant torque spring 1091 may function as a constant torque power source. As seen, the constant torque spring 1091 comprises a pulley 10944, a second spool 10942 (also referred to as output spool 10942), and a first spool 10941 (also referred to as storage spool 10941). The pulley 10944 may have a central hole 10943 and a radius, r. In some cases, the constant torque spring 1091 comprises a first portion 10945-a wound around the storage spool 10941 and a second portion 10945-b wound around the output spool 10942. That is, the constant torque spring 1091 is positioned/wrapped around the outer circumference of each of the first and second spools. As known in the art, a constant torque spring is a specially stressed constant force spring traveling between two spools. The constant torque spring is stored on a storage spool, such as storage spool 10941, and reverse-wound onto an output spool, such as output spool 10942. When released, torque is obtained from the output spool 10942 as the constant torque spring 1091 returns to its natural curvature on the storage spool 10941. It should be noted that, the constant torque spring 1091 need not be attached to the storage spool 10941. For example, in some embodiments, the constant torque spring 1091 may be housed in a cavity in the segmentation instrument, which eliminates the need for a storage spool. In one non-limiting example, the constant torque spring 1091 may be made from Type 301 stainless steel, carbon steel, or any other applicable material. As seen, each of the storage and output spools 1041 and 10942, respectively, may have a width, W. Further, the width (W) of the storage and output spools may be similar or substantially similar to the width of the constant torque springs 1091 (i.e., the width of the first and second portions 10945-a and 10945-b of the constant torque spring). Additionally, the thickness of the spring 1091, t, is shown in FIG. 10C. FIG. 10C also shows the diameter, Dₛ and Dₒ, of the first and second loop spools 10941 and 10942, respectively, of the constant torque spring 1091. The centers of the first and second portions 10945-a and 10945-b of the spring 1091 are separated by a distance, S. In this example, the distance between the spool centers is similar or substantially similar to the distance between the spring centers. In some cases, the distance, S, between the spool centers is greater than the radius of the spring when fully wound on the output spool 10942.

In some cases, a plurality of visual or electrical markers (e.g., shown as visual or electrical markers 1102 in FIG. 8) may be provided on the constant torque spring 1091. The markers may include lines (colored, or electrically isolated) placed at uniform distances along the constant torque spring 1091, and relatedly, optical or electrical sensors (shown as sensors 1106 in FIG. 8) may be provided to detect or count each time a spring marker (e.g., spring marker 1102 in FIG. 8) is encountered, and thereby infer the distance traveled by the constant torque spring 1091. In some other cases, the spring

In some other cases, the constant torque spring 1091 of FIG. 10C may be part of a reusable component of a segmentation instrument, such as, but not limited to, the reusable portion (e.g., motor 10712) previously described in relation to FIG. 9. In such cases, the constant torque spring 1091 may be rewound and reconnected to different wire loop spools (e.g., different output and storage spools).

### Electrode Wire Grasping Features

In some cases, for instance, for actuator systems which use electrode wires in combination with mechanical tension, the ability of the wire to initially grasp the surface of the tissue specimen may aid in tissue segmentation. In such cases, a lower initial pretension force (i.e., prior to actual segmentation) may be used to assist the segmenting/cutting wire in grasping the surface of the tissue specimen. According to aspects of this disclosure, surface treatments or features may be added to electrode/cutting wires (e.g., segmenting wire loop 1025 in FIG. 2, wires 10645 in FIG. 6, wires 11063 in FIGS. 12A-12C, etc.) to encourage grip between the wire(s) and the tissue specimen. In some other cases, barbs and/or other non-uniform surface features may be provided to enhance the grip between wires and tissue specimens.

Additionally, or alternatively, a coagulation or low amplitude cutting waveform may be utilized to encourage a wire to stick to (or grip) the surface of the interfacing tissue through desiccation between the tissue and wire interface. In some cases, a coagulation waveform may be used, initially, for each of the electrode wires (e.g., segmenting wire loop 1025 in FIG. 2, wires 10645 in FIG. 6, wires 11063 in FIG. 13, etc.). In other cases, coagulation waveforms may be used for only a portion of the electrode wires. In yet other cases, coagulation waveforms may be used in conjunction with the surface treatments/features described above to help the wires grip the tissue specimen. Alternatively, the wire channels holding and attaching the wires to the bag may be semi-detachable such that the wire perforation channel is attached to the bag but allowed to pull away from the bag in areas along the length of the wires. This helps hold the wires in place on the tissue until segmentation is initiated (e.g., mechanically or via application of RF energy).

The present disclosure provides devices, systems, and methods for tissue specimen removal utilizing a specimen bag and an integrated connector carrier. FIG. 11A illustrates an example of a specimen bag and connector carrier assembly 10100, according to various aspects of the disclosure. Because the specimen bag and connector carrier assembly 10100 are integrated in the embodiments shown, this may be referred to simply as the “specimen bag assembly 10100”. The specimen bag assembly 10100 comprises a specimen bag 10101 with a flexible ring that may be attached to the bag opening. The flexible ring in the embodiment shown may be made of a metal that is sufficiently thin to be flexible and have spring-like qualities. In some examples, the flexible ring comprises two separate spring arms that are coupled with a flexible member at a distal end and are held securely at a proximal end 10104. It is contemplated that the flexible ring may comprise more or fewer separate components; for example, it may be a single flexible ring, or it may have more separable parts. Though not shown, the specimen bag 10101 may comprise a plurality of segmenting components within or adjacent to its walls.

In an intermediate location between the specimen bag 10101 and a cannula assembly (not shown in FIG. 11A), a connector carrier 10105 is shown. The connector carrier 10105 performs several functions which are shown and described in subsequent figures, including holding connectors configured to attach to segmentation equipment, providing a guide to travel along the flexible ring to close or open the bag opening, providing a channel for a return electrode cable 10108 to extend out away from the bag, to secure the return electrode cable 10108 at the proximal end of the assembly to relieve forces that may be applied by pulling the return electrode cable 10108, and to provide a lock that can be integrated with a cannula or outer tube to provide a mechanical anchor at the distal most position of the outer tube. The return electrode cable 10108 may be configured to be plugged in to the piece of segmenting equipment in embodiments where the segmenting equipment is powered by RF power, as further described in relation to FIGS. 15 and 16. In such embodiments, the return electrode cable 10108, which may be attached to conductive material within the specimen bag 10101, may complete a circuit created by the segmenting equipment and the segmenting components (e.g., wire loops) within the specimen bag 10101. Embodiments of RF powered segmenting devices are shown and described throughout this disclosure.

FIG. 11B shows the connector carrier 10105 which is configured to temporarily retain the connector housing(s) 10520, according to various aspects of the disclosure. The connector housings 10520, shown in an enlarged view in FIG. 11B, are configured to connect one or more types of tissue segmentation equipment. In some cases, the connector housing 10520 and connector pins 10603 may be similar or substantially similar to the connector 11062 and pins 11061 described in relation to FIG. 12A. Additionally, or alternatively, the wire loops may implement one or more aspects of the wire loops 11063 in FIG. 12A. The connector housings 10520 are housed in the connector carrier 10105 so that the specimen bag assembly 10100 may be integrated with a variety of types of tissue segmentation components within the bag. In some cases, the connector housings 10520 may be used to manage a plurality of wire loops 10601, which are one particular type of cutting device for tissue segmentation. The wire loops may be implemented by those shown and described in U.S. Pat. Nos. 9,649,147 and 9,522,034. Any other type of cutting device may be used without departing from the scope of the present disclosure.

The connector housing 10520 may be configured such that connector pins 10603 can be extracted in only one direction (i.e., up and away from the bag, thereby pulling the wires or other cutting devices in the direction of tissue that is to be cut). These connector pins allow a plurality of wire loops 10601 (or any other type of cutting device) to be connected to additional tissue segmentation equipment. An exemplary type of tissue segmentation equipment may comprise a tensioning mechanism assembly such as the ones shown and described with reference to FIGS. 7-10C and/or 18-19.

The connectors shown can be easily connected to the tensioning mechanism assembly 10606 via a downward pressing motion onto the connectors. Then, the tension mechanism assembly 10606 may be pulled up and away from the connector carrier 10105, detaching the connector housing 10520. Then, the surgeon may move the tensioning mechanism assembly 10606 to a position directly above the center opening of the specimen bag 10101, above the specimen, and press a button on the tensioning mechanism assembly 10606 to tension the segmenting components (e.g., wire loops). In other words, the wires may be pulled taut against the surface of the tissue specimen. Because the connector pins 10603 may move independently of one another, the wires may be pulled taut against oddly shaped tissue specimens. That is, some connector pins and wires may be pulled further up into the tensioning mechanism assembly than others based on the shape of the tissue specimen a particular wire is in contact with.

The purpose of the connector housing 10520 is to retain a plurality (in this embodiment, four) of individual connection points (of, in this embodiment, wire loops) so that the user can plug in all individual connections with one plug in step. In other embodiments, there may be more connector pins per connector housing (for example, six, eight, or ten), to facilitate connections to equipment with more connection points. There may also be more connector housings 10520 than the two shown. The connector pins may also be configured in different shapes to couple with different types of equipment.

Each individual connector pin 10603 is configured to individually and independently pull away from the connector housing 10520. Each of the connector pins 10603 may therefore be manipulated separately, if necessary, to operate the connected cutting devices. If desired, the connector pins 10603 may be manually pulled and moved to facilitate manual sawing or cutting of tissue with the wire loops. In other words, the connector pins 10603 may be configured to attach to different types of tissue segmentation equipment or to none at all.

The specimen bag and cannula assembly as shown in the embodiments illustrated, have a return electrode cable 10108, which allows for the use of equipment aided with the addition of RF energy to the segmenting wires, as will be described in subsequent figures. The return electrode cable 10108 may be plugged into the RF segmentation equipment. However, the mechanism of segmentation of tissue specimen with these wires may be achieved by mechanical, electrical, or any combination of effects therein.

In the embodiment shown, the connector housing(s) 10520 connects a plurality of wire loops to a tensioning mechanism assembly in an efficient or otherwise reduced number of steps as compared to previously available mechanisms for connection to a tensioning mechanism assembly. However, the connector housing 10520 and connector pins 10603 may be used to connect to any type of multi-pin plug-in devices, such as multi-lumen tube 11052 in FIG. 12A. Alternatively, the connector pins 10603 may be used to connect mechanical, electrical, or other equipment to cutting devices. The structure of the particular connector housing 10520 shown has advantages of being able to click to allow a user to have confidence that a proper connection has been made. It also allows for the management of a plurality of wire loops or other complex segmentation components integrated within a specimen bag, and connection thereof to segmentation equipment in one step.

In order to facilitate the connector housing(s) 10520 retainment management and extraction, features may be added to the connector carrier 10105 and connector housing(s) 10520 such that the housings will be retained in place until such time when the housing is rotated (or moved) to provide an easier position for tensioning mechanism assembly connection and removal from the connector carrier 10105.

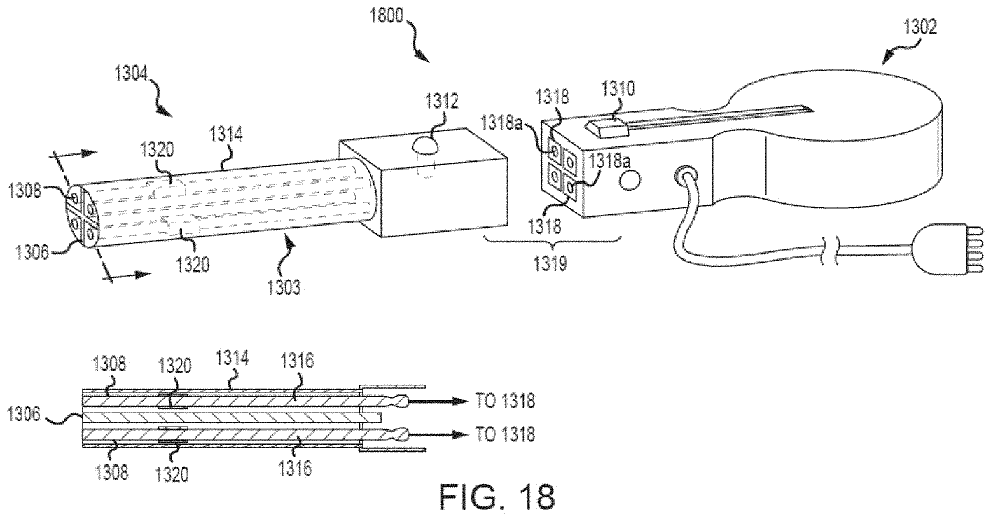

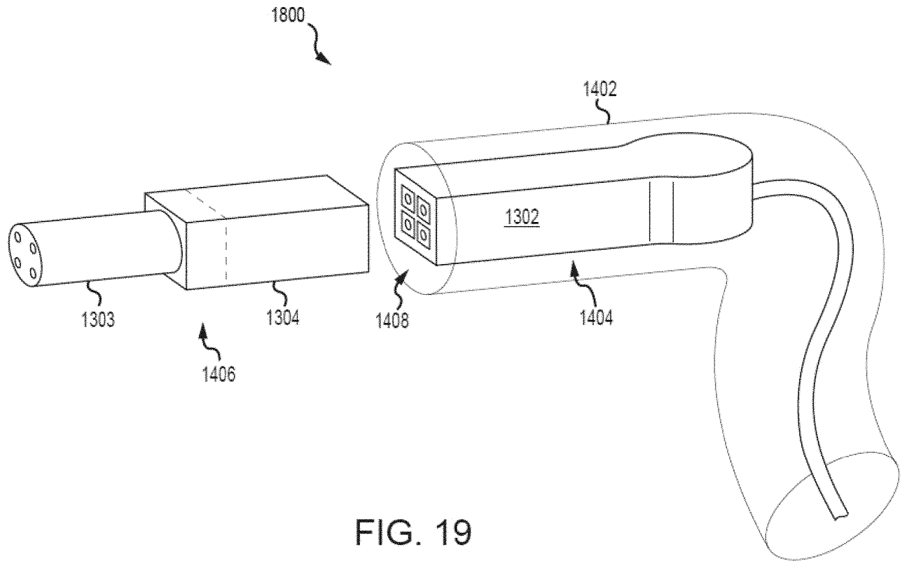

In some cases, a plurality of connector pins 10603 (also shown as pins 11061 in FIG. 12A) cover the plurality of wire loops 10601 (also shown as wire loops 11063) and are individually removable from the connector housing 10520 or connector 11062. In the embodiment shown, these connector pins 10603 themselves provide the physical connection from the wires or other segmentation components within the specimen bag 10101 to the tensioning mechanism assembly 10606, also shown and described with reference to FIGS. 18 and 19. In some cases, the tensioning mechanism or tensioning mechanism assembly in FIG. 18 may be housed within the proximal portion 1302, and the tensioning blocks 1318 may be positioned at an end, such as a distal end, of the tensioning mechanism in proximal portion 1302.

If the segmenting equipment is the tensioning device previously described, the single push of a button on the tensioning device (now plugged in) will tension each of the wire loops 10601 via the connector pins 10603, allowing the surgeon to sub-divide the tissue specimen with each of the wire loops via RF power.

### Variable Force Segmentation Instrument

Previous disclosures have identified that the RF tissue specimen removal device has an advantage in using a constant force tensioning mechanism, such as those shown and described with reference to FIG. 7, to apply the mechanical load on the segmentation wires during cutting. This method ensures that a minimum force required to perform low temperature cutting is always applied during the segmentation. The disadvantage of a constant force application is that as the tissue density and specimen sizes vary, the constant force value must be chosen to address the range of tissue variation. As such, the force value cannot be optimized for all conditions.

When RF cutting with a loop of wire wrapped around a tissue specimen with an axial mechanical load applied, the combination of mechanical and electrical energy creates a cut that initiates at the side of the tissue specimen and pulls the wire into the tissue toward the center of the specimen. This is due to the distribution of electromagnetic fields and the mechanical forces along the wire. As the segmentation advances, the cutting effect travels into the tissue and down the surface of the tissue toward the distal point. It ultimately travels to the distal most point when the wires pull completely into the tissue. As this change in wire shape occurs, the forces applied by the wire changes. The forces can be modeled as infinitesimally small segments in which each segment has a normal force into the tissue and a force axial to the wire. The location of the segment around the tissue determines the amplitude of the normal and axial force vectors. The normal force is the component that drives the wire into the tissue and performs the cutting. The axial force only advances the wire and does not significantly contribute to the cutting effect. As previously mentioned, the initiation of cutting begins in the mid-point of the tissue specimen. At this location, the normal force is at its lowest value as it is approximately 90 degrees from the axis of the applied load. As a result, the cutting begins very slowly with a small normal component. As the wire cutting advances, the change in shape and the advancement of cutting toward the distal part of the specimen increases the normal force component at the distal end of the wire. This results in a higher cutting force being applied as the segmentation advances.

One aspect of this increasing force is that the compression of the tissue due to the applied mechanical load increases during the cut. This compression may be observed by a change in the tissue impedance. At the beginning of a cut, the compression force begins at a nominal value determined by the steam pocket created around the initiated wire and the tissue impedance. As the force increases, compression of the tissue by the wire increases and the resulting impedance of the tissue reduces. This is primarily a result of the compressed tissue as well as a greater challenge for the RF energy to maintain the arcing required to sustain cutting. For most tissue specimens, this phenomenon does not have a negative impact, however with very large tissue specimens and very large applied mechanical loads, the RF energy required to sustain the cut through the end of the cut can be challenged. This effect may beneficially be considered in selection of the applied load and range of tissue compression and sizes for the system.

An alternative to a constant force, an aspect of the present disclosure relates to a variable force mechanism for applying the load to the segmentation wires. The load may be varied during the cut from a high value to a lower value to maintain a range of applied force. This approach would keep the impedance more consistent and increase the ability for the RF energy to sustain the cut.

The variable force can be applied in a linear reduction using a starting applied force and a predetermined finishing force that would be chosen to model typical tissue compression and sizes. It can also be an exponential decay to more closely model the increase in force as the wire shape changes.

An adjustable applied force may be delivered with a DC motor. This motor may be coupled to the wire with a spool such as a winch, a worm gear or with a rack and pinion that travels a length that meets or exceeds the total wire cutting length required for the largest specimen, as illustrated and described in relation to FIGS. 9-10C and 18-20C. The DC motor can be used with a current driver that can modulate the applied force based on the measured tissue impedance. In this manner, the maximum force is applied to the wire that also maintains the ability of the generator delivery power to the tissue. The DC motor may also be selected with an intrinsic load characteristic that is in line with the range of applied forces desired to allow the force delivered by the motor to be controlled with a constant current.

Turning now to FIGS. 20A-20C, in some embodiments, a tissue segmentation device 2000 (e.g., device 2000-a, device 2000-b, device 2000-c) may provide multi-wire tissue segmentation in a manner that provides a user with the ability to tension only the wire set(s) to be activated with a power, such as radio frequency (RF) energy using a RF power source 306. This ability may be helpful in isolating the entire power or RF energy application to only those wires currently involved in tissue segmentation. Specifically, those performing tissue segmentation procedures may find it helpful to have the ability to tension only wires in one planar direction, for example, all “X” direction wires for the activation of those wires, or wire sets, with the introduction of power or RF energy. These “X” direction wires may be configured to not overlap each other in physical space so as to reduce the likelihood of these active wires electrically coupling with the inactive wires. Those skilled in the art will readily envision a multitude of ways to make a mechanism 1502 which would selectively impart tensioning force to only the wire(s) to be activated, or to all wires in one planar direction.

In some embodiments, constant force springs 1503 are wound around a gear-like spool 1504 which can be locked into place, such as by a flange or tab(s) 1506 prior to tensioning or power activation.

### Reusable Segmentation Instrument

In some embodiments, RF tissue segmentation may be adapted to create a reusable portion (e.g., motor 10712 in FIG. 9, 1302 in FIG. 18) that works with a disposable portion (e.g., disposable portion 10711 in FIG. 9, 1304 in FIG. 18) of the segmentation instrument. This has the benefit of reducing overall procedure cost, as well as reducing the amount of disposed material with each use.

One embodiment of a reusable segmentation instrument described herein comprises a tensioning mechanism that utilizes a motor to apply the force. Using a motor, such as a small DC motor, has an advantage in a reusable application in that the position of the segmentation instrument tensioning mechanism can be advanced or retracted automatically. This allows easy reloading of the segmentation instrument to prepare for the next use. This reloading is much more difficult with a coil spring embodiment. In addition, the motor can be incorporated with an encoder to allow real time position information of the wire travel during cutting, and during reloading as the segmentation instrument is prepared for the next use. This allows automatic tensioning for cutting and replacement of the tensioning mechanism to the pre-load position after the segmentation is complete. Using this embodiment, the reusable portion of the device may include the electronics required for communication of the segmentation instrument to a controller, the tensioning mechanism, and the user controls. The disposable portion may be limited to the interface of the segmentation instrument with the segmentation wires.

The features and embodiments described above can be used on their own on in conjunction with and as improvements to the systems described below.

In one exemplary application, and as illustrated in FIG. 16, an advanced electrosurgical system 1600 comprising first and second wire sets 151, 160 may be provided. In this example, wire set 151 comprises electrodes/wires/wire loops 153, 155 and wire set 160 comprises electrodes/wires 157, 159. The system 1600 may be configured to perform some or all of the functions, such as tissue segmentation and/or removal, described in Applicant's International Application PCT/US15/41407, entitled Large Volume Tissue Reduction and Removal System and Method, filed on Jul. 21, 2015, and having a priority date of Jul. 22, 2014, the entire contents of which are incorporated herein by reference for all purposes, as if fully set forth herein. The system 1600 may include an electrosurgical instrument 102 and a generator 104 coupled together by a number of leads 106. The generator 104 may include a controller 108. In some embodiments, the controller 108 may be configured to cause the cutting wires/electrodes 153, 155, 157, etc., to apply radio frequency (RF) power to a tissue specimen for segmentation and removal.

Except as where otherwise stated herein, the term “segmentation device” shall be understood to include a device for dividing tissue, and may include a mechanical segmentation action, and/or an electrosurgical dissection action, for example a bipolar segmentation action, or a monopolar action.

In some embodiments, the generator 104 may include a datastore (not shown) for storing one or more sets of tissue segmentation parameters. The tissue segmentation parameters may include parameters associated with a normal or expected response during an electrosurgical procedure, and may be related to tissue segmentation voltage, current, power factor angle, impedance, power, energy, electrode or wire rate of travel, electrode or wire distance of travel, and/or mechanical segmentation force applied to tissue by the electrode(s) or wire(s). The datastore may be a component of or separate from the controller 108.

Many methods may be used to measure or determine the rate of travel. In some embodiments, and as is illustrated in FIG. 17, an optical motion sensor 674 is provided in near proximity to a spring or force application mechanism 676. The optical motion sensor may be focused on a location of the spring such that as the spring moves, the optical sensor area of focus could detect this motion as linear translation. In some embodiments, the motion may be detected as a motion within a plane.

In some embodiments, a plurality of motion sensors may be provided. The plurality of motion sensors may be configured to compare images at time T₀ against images at time T₀₊₁ to determine a direction and/or a distance of movement of the tensioning mechanism, cutting electrode, and/or wire.

In some embodiments, the sensor(s) have one or more integrated circuits, a sensor optical lens, and a light source. In some embodiments, the sensor(s) have separate components specifically for the application. The area of focus on the spring 676 may be near the spool of the spring cylinder on the flat side of the spring coil so that the movement of the spring appears as a horizontal, transverse, or ‘X’ direction motion. In some embodiments, the area of focus of the optical sensor 674 is along the extended portion of the spring away from the spring spool or cylinder. In some embodiments, the area of focus is on the top of the spool cylinder such that as the spring moves, the sensor is configured to detect rotational movement that is detected as both X and Y movement or transverse and longitudinal movement.

In some embodiments, the device may be configured to adjust a power in response to information detected and/or communicated by the sensor or plurality of sensors. For example, the device may be configured to increase a segmentation power being applied to a cutting electrode in response to a determination that the tensioning mechanism, electrode, or wire is translating or moving at a less than preferred rate. As another example, the device may be configured to decrease a segmentation power being applied to a cutting electrode in response to a determination that the tensioning mechanism, electrode, or wire is translating or moving at a greater than preferred rate.

In some embodiments, an encoder is mechanically coupled to the spring or force application mechanism to indicate a rate or distance of travel. The encoder may provide waveforms that can be used to determine a rate of travel using the phase of the two waveforms.

In some embodiments, an output of one or more sensors or a sensing circuit provides information that is used to calculate or infer a rate of travel. The electrosurgical instrument 102, which may also be referenced herein as a segmentation instrument, may use this information directly to determine if the rate of travel is acceptable. The segmentation instrument may include a processing device, an analog circuit, and/or a digital circuit to calculate, process, and/or track a sensor output. In some embodiments, the device may initiate an action responsive to the information from the one or more sensors, such as, for example only when a distance or rate of travel is outside an acceptable or expected range.

It may be beneficial to scale this information into units that are meaningful to users such as cm/second. In some embodiments, the device or controller 108 has a processor configured to scale a digital, analog, or other signal into an informative output in a manner known to those skilled in the art. One benefit of using this method is that the motion of the spring can be quantified in a traceable manner that can be compared to external measurement equipment. An additional benefit is that correction algorithms can be applied if a non-linearity is observed in the rate of travel through the entire range of travel of the spring or force application mechanism.

In some embodiments, the segmentation instrument has a controller 108 and/or a processing device in communication with the sensor(s). In some embodiments, the segmentation device may have a microprocessor, state machine, and/or field programmable gate array (FPGA) to perform the processing and/or allow a user to configure the segmentation device.

In some embodiments, a force gauge may be coupled to the tensioning mechanism assembly (e.g., tensioning mechanism assembly 10606 in FIG. 11B, tensioning mechanism or reusable portion 1071 in FIG. 9), and the power may be adjusted to assist the spring in maintaining a substantially constant force and/or a force above or below a desired threshold for suitable tissue segmentation. These methods may be used for other means of applying the tissue segmentation force, such as a linear actuator or manual pull.

In some embodiments, the controller 108 may be a box that is set on the generator 104 (also shown as RF power source 306 in FIG. 15) and has a separate power cord, or, in some embodiments, the controller 108 may be unitary with, and a component of, the generator 104, as illustrated in FIG. 16, or may be unitary with, or a component of, the electrosurgical instrument 102. The controller 108 may have only the power such as RF power connections attached to the generator 104 or may have an additional connection to communicate with a generator 104, a datastore (not shown), the electrosurgical instrument 102, and/or a user interface (not shown). This additional communication allows information to be transferred to and from the generator 104. This information may include power and mode settings, return electrode impedance information, error information such as deviation from tissue segmentation parameters as previously described herein, storage and statistical information of the procedure parameters and variables, and historical statistical information of the procedural parameter database.

The controller 108 and/or generator 104 employing the controller 108 may have the ability to measure the current I, voltage V, and/or other variables associated with the power delivered by the generator 104 prior to connecting the generator 104 output to the electrosurgical instrument 102. This allows the controller 108 to ensure that the user has selected the proper generator setting before applying electrosurgical RF energy to the wire(s)/electrode(s), to ensure that the integrity of any coating on the wire(s)/electrode(s) is maintained for initiation.

Turning now to FIG. 8, in some embodiments, various methods and systems for detecting a distance and velocity of travel of one or more wire electrodes (e.g., wire electrodes 322, 324 in FIG. 15). In some embodiments, for example, a plurality of visual or electrical markers 1102 on one or more constant force springs 1104 may be provided. The markers 1102 may include lines (colored, narrow magnetic strips, or electrically isolated) placed at uniform distances along each spring 1104, and, relatedly, optical or electrical sensor(s) 1106 may be provided to detect or count each time a spring mark 1102 is encountered, and thereby infer the distance traveled and/or rate of travel. These marks may also include a larger width that is periodically included at a different uniform distance, which serves to act as a major graduation mark. This major graduation mark may be used as a gross distance measure and/or may be used for count correction, such as if the rate of travel approaches the upper limit of the ability of the electrosurgical instrument 102 or system 1600 to measure the rate of travel. In some embodiments, the spring marks 1102 are color coded or otherwise modified verses a distance along the spring 1104, such that a color photosensor or other identifying means may determine a position of the cutting wire assembly or wires 322, 324. In some cases, the sensor 1106 may be a magnetic sensor, such as a Hall effect sensor or a Reed sensor, and the markers 1102 may be magnetized.

Continuing now with FIGS. 18-19, a reusable tissue segmentation device 1800 may be provided. The reusable tissue segmentation device 1800 may implement one or more aspects of the reusable segmentation device described in relation to FIG. 9. The reusable tissue segmentation device 1800 may be configured to perform some or all of the functions previously described herein with reference to electrosurgical instrument 102 or system 1600 previously described herein and the device described in Applicant's application PCT/US 15/41407. The reusable tissue segmentation device 1800 may be used as the connectable segmentation equipment used to connect to the connectors referenced in FIGS. 9-12C.

The device 1800 may include a proximal portion 1302 that is detachably connected or connectable to a distal portion 1304. A connection region 1319 between the proximal portion 1302 and the distal portion 1304 may be a block of a wire tensioning mechanism, such that a disposable lumen 1303 (also shown as multi-lumen tube 11052 in FIGS. 12A-12C) is attached. The disposable lumen 1303 may provide a guide 1306 for one or more tensioning mechanisms having a post 1316 that connects to tensioning blocks 1318 on the proximal portion 1302 and may have connection points to enable the distal end 1308 to connect to the active electrode wire connections (not illustrated but shown in FIG. 12A). The disposable lumen 1303 may also include a means to advance tensioning springs (or a tensioning force mechanism) to a pre-tension position, a pre-tension mechanism control 1312 that allows the user to pre-tension the tensioning mechanisms, an introducer tube 1314 for placement in the incision site, and/or a specimen bag.

With continued reference to FIGS. 18 and 19, a method of using the disposable lumen 1303 (also shown as disposable multi-lumen tube 11052 in FIG. 12A) is now described in further detail. In some embodiments, a control 1310 may be provided to allow the springs and the tensioning blocks 1318 of a proximal portion 1302 to be advanced to a distal position. The control 1310 may be a control tab. The springs and tensioning blocks 1318 may be held in a distal position by a locking mechanism (not illustrated) within the proximal portion 1302.

The user may connect the distal portion 1304 to the proximal portion 1302 by sliding the portions 1304, 1302 together such that the post(s) 1316 (see FIG. 18) in the distal portion 1304 snaps/slides/locks into receiving openings 1318a of the tensioning blocks 1318 at the end of the tensioning mechanisms in proximal portion 1302. This attachment may also cause the control 1310 or control tab to slide proximally, or back away from the distal portion 1304 and allow alignment of the pre-tension mechanism control 1312 with the locking mechanism in the proximal portion 1302. The proximal and distal portions 1302, 1304 may be configured such that pressing the pre-tension mechanism control 1312 after attachment will release the locking mechanism and pre-tension the four tensioning mechanisms. Those skilled in the art will appreciate that a number of different release methods may be provided.

Continuing with FIGS. 18 and 19, in some embodiments, the tensioning mechanisms (shown as tensioning mechanism assembly 10606 in FIG. 11B) may be connected to active electrode connectors (not illustrated) prior to pre-tensioning and may be contained within the guides 1306 during pre-tensioning and cutting.

The applied force generated by the tensioning mechanism in the proximal portion 1302 may be mechanically and electrically coupled from tensioning blocks 1318 through the posts 1316, through the alignment blocks 1320, through the distal end 1308 and through the active electrode connectors. In some embodiments, all patient contact areas may be part of a disposable lumen 1303, which may provide for simplified cleaning and reprocessing of the reusable portion including the proximal portion 1302.

In some embodiments, and as illustrated in FIG. 19, a reusable portion 1404 or reusable portions of the segmentation device may be enclosed by or carried within a sterile bag(s) 1402 with an aseptic transfer process. The sterile bag(s) 1402 may enclose the reusable portion(s) 1404, and a disposable portion 1406 may be attached to the reusable portion(s) by the user. Access through the bag may be made through an access opening 1408 in the bag 1402. In some embodiments, the access opening 1408 is open or opened behind a sleeve that can be moved, translated or folded away, and/or punctured by a feature of the disposable portion when the user connects the disposable and reusable portions. In some embodiments, a sterile adapter is integrated into the sterile bag(s) 1402 to facilitate connection of the sterile disposable portion(s) of the device and the non-sterile reusable portion(s), while retaining sterility in the sterile field. Those skilled in the art will readily recognize a number of means of providing a reusable portion(s) 1404 and a disposable portion(s) 1406 and enabling connection of the portions. Any and all means now known or as yet to be developed are contemplated herein.

Some embodiments providing means for separating the reusable components from the patient contact components may include a disposable insert inside the reusable tissue segmentation device 1800. The disposable insert may capture the wires after the cut. In some embodiments, a device that can be easily disassembled so that the interior area that contains the wires after the cut can be cleaned, reassembled and re-sterilized.

In some embodiments, a tensioning mechanism may include a constant force spring 1091 and/or other mechanisms such as a pulley system (e.g., pulley 10944), a cable drive or winch system, non-linear springs, linear drive with rotational coupling such as gears or contact coupling, linear drive with magnetic coupling, linear drive with manual control, and/or, as previously described, an electromechanical drive, such as a servo or stepper motor drive or linear actuator.

As illustrated in FIG. 21, which depicts an example of a spring system 2100 for tensioning segmenting wires, torsion springs 8302 for achieving wire tension during a cut may be provided. The torsion springs 8302 may be constant force springs and may provide for the retraction of the cutting wires, electrodes or wire loops. The torsion springs may coil the wire or other structure that pulls the wire into the device shaft. The torsion spring 8302 may operate sequentially.

As illustrated in FIG. 14, in some cases, a robotic or other electromechanical means may be utilized for a surgery. In such cases, it may be desired to utilize the same means to remove the segments from the bag. FIG. 14 illustrates another example 1400 of a tissue specimen bag deployed inside a cavity of a patient and a grasper, according to various aspects of the present disclosure. FIG. 14 illustrates an exemplary approach to enabling robotic assisted removal. As illustrated, a system 8830 having a tissue removal bag 8831, a robotic grasper 8832, a guide means 8834, and a bag-machine interface 8836 is provided in some embodiments. In some cases, the grasper 8832 implements one or more aspects of the grasper(s) described in relation to FIGS. 1-6.

The robotic grasper 8832 may include a camera and/or a light source 8839 on an arm 8835 to allow a surgeon to view the robotic grasper 8832 going in and out of a patient's body or incision. The guide means 8834 provides the ability to guide the robotic grasper 8832 in and out of the incision or a trocar including a guide between the trocar or incision site. In some embodiments the robotic grasper 8832 is configured to travel between the incision site and another location (such as a specimen or pathology container, or a tray to receive tissue).

The bag-machine interface 8836 may be provided on or proximal to the bag opening and is configured to interface with a robotic arm 8838 and allow the arm 8838 to provide tension on the bag 8831 during removal of the tissue segments 8822 such that the segments are easily identified and grasped.

Although this document primarily addresses electrosurgical systems, it should be understood that tissue segmentation and removal may, in some embodiments, but achieved using a segmentation device that does not have an electrosurgical component. Specifically, a surgical device having one or more wires that segment tissue mechanically, such as by force, motion, and/or vibration may be provided. Many of the examples disclosed herein also apply to such a mechanical surgical device. For example, a surgical device may utilize wire tensioning methods disclosed herein without the electrical aspects, and with or without a controller configured to control the pull forces or speed of cut. Similarly, the robotic system may also provide a cutting function that is not electrosurgical in nature. As in the case of the electrosurgical segmentation procedure, the removal bag may provide means for keeping the cutting wires in place (and from entangling with each other) while a tissue segment is placed in the removal bag, and, similarly, the wires may be configured to detach from the removal bag at a desired set force or time. The use of mechanical only cutting may be advantageous in applications where the tissues are not calcified, have less variability of mechanical properties, or are generally more friable, and therefore do not require extremely high forces to cut reliably through the tissues. To address this case, the tissue removal device or wire cutting device may be configured without the elements that are required for electrosurgical cutting; for example, the return electrode or connections to the controller or an electrosurgical generator may be omitted. Those skilled in the art will understand that a removal device without the electrosurgical cutting elements requires a smaller number of user completed instrument connections. In turn, this may lower the production costs of the product. In some embodiments, a removal device that does not have an electrosurgical cutting feature allows for cutting tissue at a lower temperature, and may be a safer alternative for weaker patients. Those skilled in the art will understand that the mechanical pull force(s) in a removal device without electrosurgical cutting will be significantly greater than one with an electrosurgical cutting feature.

Each of the various elements disclosed herein may be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, the disclosure of a “cutting mechanism” should be understood to encompass disclosure of the act of “cutting”—whether explicitly discussed or not—and, conversely, were there only disclosure of the act of “cutting”, such a disclosure should be understood to encompass disclosure of a “cutting mechanism”. Such changes and alternative terms are to be understood to be explicitly included in the description.

The previous description of the disclosed embodiments is provided to enable any person skilled in the art to make or use the present invention defined by the claims. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A tissue segmentation device, comprising:
one or more segmenting wires;
a grasper;
an introducer tube having a proximal end and a distal end, wherein the introducer tube is shaped and sized to allow introduction of the one or more segmenting wires and the grasper into an incision in a patient; and
at least one actuator positioned at or near the proximal end of the introducer tube, wherein the at least one actuator is coupled to a proximal portion of the one or more segmenting wires and a proximal portion of the grasper, and wherein the at least one actuator is configured for manipulating the grasper to grasp a tissue specimen prior to or during tissue segmentation;
wherein manipulation of the grasper further enables one or more of:
pulling the tissue specimen into the one or more segmenting wires for segmenting said tissue specimen; and
positioning the tissue specimen such that it contacts the one or more segmenting wires.

2. The tissue segmentation device of claim 1, wherein the one or more segmenting wires comprise a plurality of segmenting wires, and wherein at least one of the plurality of segmenting wires is an active electrode configured to carry radio frequency (RF) energy.

3. The tissue segmentation device of claim 2, wherein the active electrode is a stationary electrode, and the grasper comprises a return electrode, and wherein the manipulation of the grasper comprises pulling the tissue specimen into the active electrode for segmentation of said tissue specimen.

4. The tissue segmentation device of claim 2, wherein the at least one actuator is configured to expand the active electrode into a bulbous loop shape adjacent to, but not in contact with, a return electrode, and wherein the grasper comprises the return electrode.

5. The tissue segmentation device of claim 2, wherein,
at least a portion of the grasper is conductive,
the grasper comprises a return electrode,
the active electrode comprises a single active electrode, and
a surface area of the return electrode is greater than a surface area of the single active electrode.

6. The tissue segmentation device of claim 1, wherein the one or more segmenting wires comprises a plurality of segmenting wires, the plurality of segmenting wires shaped and sized to fit within an inner diameter of the introducer tube.

7. The tissue segmentation device of claim 6, wherein the plurality of segmenting wires comprise an expanded position and a retracted position, and wherein,
when in the expanded position, the plurality of segmenting wires are configured to extend at an angle from the distal end of the introducer tube, and
when in the retracted position, the plurality of segmenting wires are parallel or substantially parallel to each other and configured to retract into the distal end of the introducer tube.

8. The tissue segmentation device of claim 7, wherein, when in the expanded position, the plurality of segmenting wires are configured to segment the tissue specimen upon one of:
(1) pulling the tissue specimen into the plurality of segmenting wires using the grasper, wherein the grasper comprises a return electrode, and wherein one or more of the plurality of segmenting wires comprise an active electrode, or
(2) pushing the plurality of segmenting wires into the tissue specimen, wherein one or more of the plurality of segmenting wires comprise an active electrode.

9. The tissue segmentation device of claim 1, wherein the one or more segmenting wires comprises a plurality of segmenting wire loops, and wherein positioning the tissue specimen further comprises:
encircling at least a portion of the tissue specimen using the plurality of segmenting wire loops.

10. The tissue segmentation device of claim 1, further comprising:
a plurality of retractable tines configured to expand from and retract into the distal end of the introducer tube, wherein at least one of the plurality of retractable tines is a return electrode and at least two of the plurality of retractable tines are active electrodes,
wherein the return electrode is arranged opposing the active electrodes such that the return electrode does not contact the active electrodes.

11. A tissue segmentation device, comprising:
one or more wire loop spools;
one or more segmenting wires, wherein at least a portion of each of the one or more segmenting wires is wound on one of the one or more wire loop spools; and
a tensioning mechanism comprising at least one motor, wherein the at least one motor of the tensioning mechanism is coupled to the one or more wire loop spools and configured to provide an adjustable force to advance or retract the one or more segmenting wires via a corresponding wire loop spool.

12. The tissue segmentation device of claim 11, wherein the one or more segmenting wires comprise a plurality of segmenting wire loops, the tissue segmentation device further comprising:
an introducer tube having a proximal end and a distal end, wherein the introducer tube is shaped and sized to allow introduction of the one or more segmenting wires into an incision in a patient;
a multi-lumen tube comprising a plurality of lumens or channels, the multi-lumen tube shaped and sized to fit within an inner diameter of the introducer tube; and
a plurality of connector pins coupled to ends of the plurality of segmenting wire loops;
wherein each of the plurality of connector pins is received within one lumen or channel of the multi-lumen tube.

13. The tissue segmentation device of claim 12, further comprising:
a connector for reducing or minimizing friction between the plurality of segmenting wire loops and the multi-lumen tube,
wherein the connector is positioned at or near a distal end of the multi-lumen tube, and
wherein the plurality of connector pins are positioned on a proximal portion of the connector.

14. The tissue segmentation device of claim 12, wherein the multi-lumen tube further comprises a rod, the rod shaped and sized to be received within a lumen or channel of the multi-lumen tube, and wherein a central axis of the rod is positioned at or near a central axis of the multi-lumen tube.

15. The tissue segmentation device of claim 11, wherein each of the one or more wire loop spools comprises a slot that is shaped and sized to receive a rotating paddle from one of the at least one motor, and wherein each of the at least one motor is configured to provide an adjustable force to one of the one or more segmenting wires via a corresponding wire loop spool.

16. The tissue segmentation device of claim 11, wherein each of the one or more segmenting wires is an active electrode configured to receive a radio frequency (RF) signal from a RF generator, and wherein each of the one or more wire loop spools comprises a conductive metal disk and a drag strip connection for electrically coupling the RF generator to a corresponding one of the one or more segmenting wires via the at least one motor.

17. The tissue segmentation device of claim 11, wherein the tensioning mechanism is coupled to a pneumatic system, the pneumatic system configured to generate pressure that is above a threshold for driving a translation force for advancing or retracting the one or more segmenting wires.

18. A tissue segmentation device, comprising:
a disposable portion comprising one or more wire loop spools, wherein a segmenting wire is wound around each of the one or more wire loop spools; and
a reusable portion, the reusable portion comprising at least a tensioning mechanism assembly, wherein the tensioning mechanism assembly is configured to couple to each of the one or more wire loop spools, and wherein the tensioning mechanism assembly is further configured for applying tension to the segmenting wire via rotation of the one or more wire loop spools.

19. The tissue segmentation device of claim 18, wherein the tensioning mechanism assembly comprises at least a motor or a spring, wherein the motor is a direct current (DC) motor, and the spring is a constant torque or constant force spring.

20. The tissue segmentation device of claim 18, further comprising:
at least one controller, the at least one controller configured to control one or more of:
a power output of a radio frequency (RF) generator, the RF generator configured to supply RF energy or power to the segmenting wire; and
a torque or force applied by a force application mechanism of the tensioning mechanism assembly to the segmenting wire, based at least in part on determining one or more of:
a rate of travel of the force application mechanism,
a distance of travel of the force application mechanism,
a rate of travel of each of the segmenting wire, and
a distance of travel of the segmenting wire.
